(19) United States
(12) Patent Application Publication
(10) Pub. No.: US 2024/0130371 A1
(43) Pub. Date: Apr. 25, 2024

(54) ENVIRONMENTAL PROTECTION SYSTEMS AND METHODS OF GREENHOUSE GAS CAPTURE VIA CALCIUM HYDROXIDE

(71) Applicants: ROSHDY RADY ATEYA SOLIMAN, OTTAWA (CA); RAFIK ROSHDY RADY ATEYA, OTTAWA (CA); RAFFAELLA ROSHDY RADY ATEYA, OTTAWA (CA); INGI ROSHDY RADY ATEYA, OTTAWA (CA)

(72) Inventors: ROSHDY RADY ATEYA SOLIMAN, OTTAWA (CA); RAFIK ROSHDY RADY ATEYA, OTTAWA (CA); RAFFAELLA ROSHDY RADY ATEYA, OTTAWA (CA); INGI ROSHDY RADY ATEYA, OTTAWA (CA)

(21) Appl. No.: 18/546,218

(22) PCT Filed: Feb. 10, 2022
(86) PCT No.: PCT/CA2022/050188

§ 371 Date: Aug. 11, 2023

Related U.S. Application Data
(60) Provisional application No. 63/148,700, filed on Feb. 12, 2021.

(51) Int. Cl. A01N 59/06; A01N 3/02; A01P 1/00; A23B 7/148; A23B 7/154; A23B 7/157; A23B 7/16; A23B 9/20; A23B 9/30; A23K 20/24; B01D 53/92; B65B 25/04; B65B 55/00; C05D 3/02; C05F 3/00; C05F 7/00; C05F 9/04; C05G 5/27; C10L 1/12; C12P 7/54

(52) U.S. Cl. CPC A01N 59/06; A01N 3/02; A01P 1/00; A23B 7/148; A23B 7/154; A23B 7/157; A23B 7/16; A23B 9/20; A23B 9/30; A23K 20/24; B01D 53/92; B65B 25/041; B65B 55/00; C05D 3/02; C05F 3/00; C05F 7/005; C05F 9/04; C05G 5/27; C10L 1/1233; C12P 7/54; B01D 2251/2067; B01D 2251/404; B01D 2251/604; B01D 2257/302; B01D 2257/404; B01D 2257/504; B01D 2258/012

(57) ABSTRACT

Multiple environmental, economic, and ecological issues are linked to a limited number of chemicals such as carbon dioxide, sulphur dioxide, methane, nitrous oxide, methanol, formaldehyde and ethylene or disposal of some waste products from farming, transportation, and the beverage industry. Accordingly, the reduction of the impacts of these limited number of gases on the environment and/or ecology is beneficial. Accordingly, there are presented a range of application of methods, systems, and treatments which mitigate these impacts.

### CROSS-REFERENCE TO RELATED APPLICATIONS

This patent application claims the benefit of priority as a 371 National Phase entry application of PCT/CA2022/050188 filed Feb. 10, 2022; which itself claims the benefit of priority from U.S. Provisional Patent application 63/148,700 filed Feb. 12, 2021; the entire contents of each being incorporated herein by reference.

### FIELD OF THE INVENTION

This patent application relates to the environment and more particularly to systems and methods for protection of the environment and products.

### BACKGROUND OF THE INVENTION

Multiple environmental, economic and ecological issues are linked to a limited number of chemicals such as carbon dioxide, sulphur dioxide, methane, nitrous oxide, methanol, formaldehyde and ethylene or the disposal of some waste products from farming, transportation and the beverage industry. It would therefore be beneficial to reduce the impacts of these limited number of gases through the application of methods, systems, and treatments to absorb these gases.

Other aspects and features of the present invention will become apparent to those ordinarily skilled in the art upon review of the following description of specific embodiments of the invention in conjunction with the accompanying figures.

### SUMMARY OF THE INVENTION

It is an object of the present invention to mitigate limitations within the prior art relating to environment and more particularly to systems and methods for protection of the environment and products.

In accordance with an embodiment of the invention there is provided a method comprising executing a first process with respect to a quantity of produce, wherein the first process delays at least one of a spoiling and a ripening of the produce.

In accordance with an embodiment of the invention there is provided a method comprising:
applying a solution to at least one of a fruit, a vegetable and an herb; wherein
the solution is a calcium hydroxide solution;
the solution absorbs carbon dioxide in the vicinity of the at least one of the fruit, the vegetable and the herb via chemical reaction generating at least calcium carbonate; and
the solution delays at least one of a spoiling and a ripening of the at least one of the fruit, the vegetable and the herb.

In accordance with an embodiment of the invention, there is provided a container comprising:
a casing;
a permeable membrane forming a predetermined portion of the casing; and
calcium hydroxide powder within the container.

In accordance with an embodiment of the invention there is provided a method comprising:
providing a container comprising a casing, a permeable membrane forming a predetermined portion of the casing and calcium hydroxide powder within the container;
placing the container in predetermined relationship to at least one of a fruit, a vegetable and an herb; wherein
the solution absorbs carbon dioxide in the vicinity of the at least one of the fruit, the vegetable and the herb via chemical reaction generating at least calcium carbonate; and
the solution delays at least one of a spoiling and a ripening of the at least one of the fruit, the vegetable and the herb.

In accordance with an embodiment of the invention there is provided a method comprising:
at least one of storing and shipping at least one of a plurality of seeds and a plurality of seedlings with container; wherein
the container comprises:
   a casing;
   a permeable membrane forming a predetermined portion of the casing; and
   at least one of:
      calcium hydroxide powder disposed within the container; and
      calcium hydroxide powder and activated carbon powder within the container;
the calcium hydroxide absorbs at least one of moisture and carbon dioxide within the vicinity of the plurality of seeds and the plurality of seedlings; and
the activated carbon if present acts in combination with the calcium hydroxide powder to absorb at least one carbon dioxide and an odour.

In accordance with an embodiment of the invention there is provided a method comprising:
providing an organic byproduct;
providing an additive comprising a calcium hydroxide solution and coffee grounds in a first predetermined ratio;
adding the additive to the organic byproduct at a second predetermined ratio; and
applying the organic byproduct with additive to soil.

In accordance with an embodiment of the invention there is provided a cleaning method comprising:
applying a calcium hydroxide solution to a predetermined region of an indoor environment; wherein
the calcium hydroxide solution acts as an absorber of carbon dioxide within the indoor environment and at least one of an antimicrobial agent and an antiviral agent.

In accordance with an embodiment of the invention there is provided a method comprising:
(a) acquiring carbon dioxide;
(b) reacting the acquired carbon dioxide with hydrogen to generate formic acid;
(c) acquiring methane gas from organic byproducts breaking down;
(d) reacting the methane with formic acid in the presence of heat and a bacterial catalyst; and
(e) capturing hydrogen generated from step (d);
(f) extracting the acetic acid generated from step (d).

In accordance with an embodiment of the invention there is provided a solution for combination with diesel exhaust gases comprising urea, water, and calcium hydroxide in a predetermined ratio.

In accordance with an embodiment of the invention there is provided a method of reducing emissions from a diesel engine comprising injecting a solution at specific flow rate into an exhaust of the diesel engine when the diesel engine is running, wherein the solution comprises urea, water and calcium hydroxide.

In accordance with an embodiment of the invention there is provided a method comprising:
at least one of extending freshness and delaying ripening of produce with calcium hydroxide; wherein
the calcium hydroxide is applied by at least one of a spraying a calcium hydroxide solution onto the produce; misting the calcium hydroxide solution onto the produce, dipping the produce into the calcium hydroxide solution; storing the produce with calcium hydroxide powder, and coating the produce with calcium hydroxide powder.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures, wherein:

FIGS. 1A and 1B depict exemplary images of bananas showing reduced spoiling/ripening from application of a solution according to an embodiment of the invention;

FIGS. 2A and 2B depict exemplary images of beetroots showing reduced spoiling/ripening from application of a solution according to an embodiment of the invention;

FIGS. 3A and 3B depict exemplary images of parsley showing reduced spoiling/ripening from application of a solution according to an embodiment of the invention;

FIGS. 4A and 4B depict exemplary images of tomatoes showing reduced spoiling/ripening from application of a solution according to an embodiment of the invention;

FIGS. 5A and 5B depict exemplary images of apples showing reduced spoiling/ripening from storage within a container with a housing containing a powder according to an embodiment of the invention;

FIGS. 6A and 6B depict exemplary images of aubergines (eggplants) showing reduced spoiling/ripening from storage within a container with a housing containing a powder (referred to as a Product Package) according to an embodiment of the invention;

FIG. 7 depicts the short term evolution of carbon dioxide levels of packaged and unpackaged apples with and without a Product Package according to an embodiment of the invention;

FIGS. 8A and 8B depict packaged and unpackaged apples with and without a Product Package according to an embodiment of the invention with a sensor device depicting the carbon dioxide levels;

FIG. 9 depicts the short term evolution of carbon dioxide levels of packaged and unpackaged red peppers with and without a Product Package according to an embodiment of the invention;

FIGS. 10A and 10B depict packaged and unpackaged red peppers with and without a Product Package according to an embodiment of the invention with a sensor device depicting the carbon dioxide levels;

FIG. 11 depicts the short term evolution of carbon dioxide levels of packaged and unpackaged red potatoes with and without a Product Package according to an embodiment of the invention;

FIGS. 12A and 12B depict packaged and unpackaged red potatoes with and without a Product Package according to an embodiment of the invention with a sensor device depicting the carbon dioxide levels;

FIG. 13 depicts the short term evolution of carbon dioxide levels of packaged and unpackaged red onions with and without a Product Package according to an embodiment of the invention;

FIGS. 14A and 14B depict packaged and unpackaged red onions with and without a Product Package according to an embodiment of the invention with a sensor device depicting the carbon dioxide levels;

FIG. 15 depicts the short term evolution of carbon dioxide levels of packaged and unpackaged yellow onions with and without a Product Package according to an embodiment of the invention;

FIGS. 16A and 16B depict packaged and unpackaged yellow onions with and without a Product Package according to an embodiment of the invention with a sensor device depicting the carbon dioxide levels;

FIG. 17 depicts the short term evolution of carbon dioxide levels of packaged and unpackaged garlic with and without a Product Package according to an embodiment of the invention;

FIGS. 18A and 18B depict packaged and unpackaged garlic with and without a Product Package according to an embodiment of the invention with a sensor device depicting the carbon dioxide levels;

FIG. 19 depicts the short term evolution of carbon dioxide levels of packaged and unpackaged mangos with and without a Product Package according to an embodiment of the invention;

FIGS. 20A and 20B depict packaged and unpackaged mangos with and without a Product Package according to an embodiment of the invention with a sensor device depicting the carbon dioxide levels;

FIG. 21 depicts the impact of soil pH upon nutrient uptake by plants;

FIG. 22 depicts a basil plant prior to the application of 5 tablets according to an embodiment of the invention comprising 9 grams of manure, 3 grams of coffee grounds and 0.01 grams of calcium hydroxide in powdered form;

FIGS. 23 and 24 depict photographs of pellets formed according to embodiments of the invention;

FIGS. 25 and 26 depict photographs of a rose plant prior to and after application of coffee based pellets according to an embodiment of the invention;

FIG. 27 depicts photographs of basil plants with different pellets according to embodiments of the invention;

FIG. 28 depicts carbon dioxide concentration versus time after application of a solution according to an embodiment of the invention to a floor.

FIG. 29 depicts a schematic of a diesel engine exhaust processing system according to an embodiment of the invention; and

FIG. 30 depicts the reduction of carbon dioxide levels from a caulking material when employed in conjunction with a solution or powder absorber according to an embodiment of the invention

FIG. 31 depicts an exemplary process flow for the generation of acetic acid according to an embodiment of the invention;

FIG. 32 depicts photographs of roses 1 week after purchase without any additional treatment;

FIG. 33 depicts photographs of roses 1 week after purchase with a Product Package according to an embodiment of the invention placed in the vicinity of the roses;

FIG. 34 depicts photographs of roses 1 week after purchase where paper wrapping around the roses was soaked with a solution according to an embodiment of the invention

### DETAILED DESCRIPTION

The present invention is direct to environment and more particularly to systems and methods for protection of the environment and products.

The ensuing description provides representative embodiment(s) only, and is not intended to limit the scope, applicability or configuration of the disclosure. Rather, the ensuing description of the embodiment(s) will provide those skilled in the art with an enabling description for implementing an embodiment or embodiments of the invention. It being understood that various changes can be made in the function and arrangement of elements without departing from the spirit and scope as set forth in the appended claims. Accordingly, an embodiment is an example or implementation of the inventions and not the sole implementation. Various appearances of “one embodiment,” “an embodiment” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention can also be implemented in a single embodiment or any combination of embodiments.

Reference in the specification to “one embodiment,” “an embodiment,” “some embodiments” or “other embodiments” means that a particular feature, structure, or characteristic described in connection with the embodiments is included in at least one embodiment, but not necessarily all embodiments, of the inventions. The phraseology and terminology employed herein is not to be construed as limiting but is for descriptive purpose only. It is to be understood that where the claims or specification refer to “a” or “an” element, such reference is not to be construed as there being only one of that element. It is to be understood that where the specification states that a component feature, structure, or characteristic “may,” “might,” “can” or “could” be included, that particular component, feature, structure, or characteristic is not required to be included.

Reference to terms such as “left,” “right,” “top,” “bottom,” “front” and “back” are intended for use in respect to the orientation of the particular feature, structure, or element within the figures depicting embodiments of the invention. It would be evident that such directional terminology with respect to the actual use of a device has no specific meaning as the device can be employed in a multiplicity of orientations by the user or users.

Reference to terms “including,” “comprising,” “consisting” and grammatical variants thereof do not preclude the addition of one or more components, features, steps, integers or groups thereof and that the terms are not to be construed as specifying components, features, steps or integers. Likewise, the phrase “consisting essentially of,” and grammatical variants thereof, when used herein is not to be construed as excluding additional components, steps, features integers or groups thereof but rather that the additional features, integers, steps, components or groups thereof do not materially alter the basic and novel characteristics of the claimed composition, device or method. If the specification or claims refer to “an additional” element, that does not preclude there being more than one of the additional element.

“Packaged” as used herein refers to produce which has been enclosed within exterior packaging which surrounds the produce. Accordingly, such exterior packaging may comprise, but not be limited to, produce within a hard or rigid package or container such as one formed from a hard or rigid plastic, cardboard or plastic coated cardboard for example or within a soft or flexible package or container such as formed from a flexible plastic, a thin film of plastic, paper, plastic coated paper, or a re-sealable plastic bag for example.

A “Product Package” as used herein refers to a physical housing according to an embodiment of the invention. For example, a Product Package may be as described and depicted in FIG. 6 with respect to Housing 610 and as depicted within FIGS. 6A, 8B, 10B, 12B, 14B, 16B, 18B, 20B, and 33.

A “fluid” as used herein refers to a liquid, a gas, a mixture of liquids or a mixture of gases.

A “solution” as used herein refers to a homogeneous mixture composed of two or more substances comprising at least one solute dissolved in at least one solvent.

A “powder” as used herein refers to a dry, bulk solid composed of many fine particles that may flow freely when shaken or tilted. The powder may be of a single material or multiple materials.

“Urea” as used herein refers to a carbamide, an organic compound with chemical formula CO(NH₂)₂.

Humic acid, as used herein, when viewed on the basis of an acid-base theory, refers to organic acids which are organic substances extracted from soil that coagulate to form particulates when a strong-base extract is acidified. Related to humic acids are fulvic acids that are organic acids that remain soluble when a strong-base extract is acidified.

A “wireless standard” as used herein and throughout this disclosure, refer to, but is not limited to, a standard for transmitting signals and/or data through electromagnetic radiation which may be optical, radio-frequency (RF) or microwave although typically RF wireless systems and techniques dominate. A wireless standard may be defined globally, nationally, or specific to an equipment manufacturer or set of equipment manufacturers. Dominant wireless standards at present include, but are not limited to IEEE 802.11, IEEE 802.15, IEEE 802.16, IEEE 802.20, UMTS, GSM 850, GSM 900, GSM 1800, GSM 1900, GPRS, ITU-R 5.138, ITU-R 5.150, ITU-R 5.280, IMT-1000, Bluetooth, Wi-Fi, Ultra-Wideband and WiMAX. Some standards may be a conglomeration of sub-standards such as IEEE 802.11 which may refer to, but is not limited to, IEEE 802.1a, IEEE 802.11b, IEEE 802.11g, or IEEE 802.11n as well as others under the IEEE 802.11 umbrella.

A “wired standard” as used herein and throughout this disclosure, generally refer to, but is not limited to, a standard for transmitting signals and/or data through an electrical cable discretely or in combination with another signal. Such wired standards may include, but are not limited to, digital subscriber loop (DSL), Dial-Up (exploiting the public switched telephone network (PSTN) to establish a connection to an Internet service provider (ISP)), Data Over Cable Service Interface Specification (DOCSIS), Ethernet, Gigabit home networking (G.hn), Integrated Services Digital Network (ISDN), Multimedia over Coax Alliance (MoCA), and Power Line Communication (PLC, wherein data is overlaid to AC/DC power supply). In some embodiments a “wired standard” may refer to, but is not limited to, exploiting an optical cable and optical interfaces such as within Passive Optical Networks (PONs) for example.

A “sensor” as used herein may refer to, but is not limited to, a transducer providing an electrical output generated in dependence upon a magnitude of a measure and selected from the group comprising, but is not limited to, environmental sensors, medical sensors, biological sensors, chemical sensors, ambient environment sensors, position sensors, motion sensors, thermal sensors, infrared sensors, visible sensors, RFID sensors, and medical testing and diagnosis devices.

A “portable electronic device” (PED) as used herein and throughout this disclosure, refers to a wireless device used for communications and other applications that requires a battery or other independent form of energy for power. This includes devices, but is not limited to, such as a cellular telephone, smartphone, personal digital assistant (PDA), portable computer, pager, portable multimedia player, portable gaming console, laptop computer, tablet computer, a wearable device and an electronic reader.

A “fixed electronic device” (FED) as used herein and throughout this disclosure, refers to a wireless and /or wired device used for communications and other applications that requires connection to a fixed interface to obtain power. This includes, but is not limited to, a laptop computer, a personal computer, a computer server, a kiosk, a gaming console, a digital set-top box, an analog set-top box, an Internet enabled appliance, an Internet enabled television, and a multimedia player.

A “server” as used herein, and throughout this disclosure, refers to one or more physical computers co-located and/or geographically distributed running one or more services as a host to users of other computers, PEDs, FEDs, etc. to serve the client needs of these other users. This includes, but is not limited to, a database server, file server, mail server, print server, web server, gaming server, or virtual environment server.

An “application” (commonly referred to as an “app”) as used herein may refer to, but is not limited to, a “software application,” an element of a “software suite,” a computer program designed to allow an individual to perform an activity, a computer program designed to allow an electronic device to perform an activity, and a computer program designed to communicate with local and/or remote electronic devices. An application thus differs from an operating system (which runs a computer), a utility (which performs maintenance or general-purpose chores), and a programming tools (with which computer programs are created). Generally, within the following description with respect to embodiments of the invention an application is generally presented in respect of software permanently and/or temporarily installed upon a PED and/or FED and/or wearable device.

A “wearable device” or “wearable sensor” relates to miniature electronic devices that are worn by the user including those under, within, with or on top of clothing and are part of a broader general class of wearable technology which includes “wearable computers” which in contrast are directed to general or special purpose information technologies and media development. Such wearable devices and/or wearable sensors may include, but not be limited to, smartphones, smart watches, e-textiles, smart shirts, activity trackers, smart glasses, environmental sensors, medical sensors, biological sensors, physiological sensors, chemical sensors, ambient environment sensors, position sensors, neurological sensors, drug delivery systems, medical testing and diagnosis devices, and motion sensors.

A “profile” as used herein, and throughout this disclosure, refers to a computer and/or microprocessor readable data file comprising data relating to settings and/or limits of an adult device. Such profiles may be established by a manufacturer/supplier/provider of a device, service, etc. or they may be established by a user through a user interface for a device, a service or a PED/FED in communication with a device, another device, a server or a service provider etc.

Within the following description specific ratios of the constituents of compositions, formulations, mixtures, powders, and solutions of embodiments of the invention are provided. However, it would be evident to one of skill in the art that other ratios of the constituents of compositions, formulations, mixtures, powders, and solutions of embodiments of the invention of said compositions, mixtures and solutions may be employed without departing from the scope of the invention. The scope of the invention is defined by the claims.

### 1. Delaying Spoilage and Onset of Ripening in Vegetables and Fruits

An estimated 1.3 billion tonnes of food is wasted globally, a third of all food produced for human consumption. Food loss is often used to refer to food lost in earlier stages of production such as harvest, storage and transportation. In contrast, food waste is often used to refer to items that are fit for human consumption but are thrown away, often at supermarkets or by consumers. According to the Food and Agriculture Organization of the United Nations high- and low-income countries discard similar amounts of food, approximately 670 and 630 million tonnes, respectively, although there is a major difference in where and how that loss occurs.

Within low-income countries, loss occurs more often in the earlier stages. For example, in Sub-Saharan Africa, 83 per cent of food is lost during production, handling/storage and processing, while just five per cent is wasted by consumers. Conversely, in North America, Europe and Oceania, 32 per cent is lost in the earlier stages, and 61 per cent is wasted by consumers. In high income countries approximately half of the food wasted is fruit and vegetables, e.g. in Canada 15% fruit and 30% vegetables.

In economic figures, this wasted and lost food represents approximately US $2.6 trillion per annum. Accordingly, it is evident that even relatively modest reductions in the food loss/waste can have massive economic and societal impacts. For the latter, this reduced food waste may allow food to help reduce the approximately 800 million hungry people in the world.

Many fruits and vegetables exhibit accelerated ripening and subsequent spoilage through exposure to carbon dioxide. Accordingly, it would be beneficial to reduce this ripening rate with a method and system that is simple to implement, compatible with existing food storage, transportation, and display systems, and safe for human food.

Ethylene gas (C2H4) is a gas released by some fruits and vegetables during all stages of its lifecycle from farming, during transportation, storage, retail store shelves, and finally in the consumer's home. Ethylene is known as the “fruit-ripening hormone” where every fruit or vegetable has a certain level of ethylene production throughout its lifecycle. However, in some fruits or vegetables, the ethylene levels rise substantially when the fruit or vegetable starts ripening.

Similarly, formaldehyde (CH2O) is a colorless, flammable gas at room temperature with a strong odor which is also produced in small amounts by most living organisms as part of normal metabolic processes. Initially formaldehyde is generated from a reaction between methanol and oxygen but then itself reacts with oxygen to generate carbon dioxide and water as outlined below in Equations (1A) through (2B). Equations (3A) and (3B) presenting the reaction of ethylene with oxygen to generate carbon dioxide and water.

Methanol+Oxygen→Formaldehyde+Hydrogen Peroxide (1A)

CH₃OH+O₂→CH₂O+H₂O₂ (1B)

Formaldehyde+Oxygen→Carbon Dioxide+Water (2A)

CH₂O+O₂→CO₂+H₂O (2B)

Ethylene+Oxygen→Carbon Dioxide+Water (3A)

C₂H₄+3O₂→2CO₂+2H₂O (3B)

Both ethylene and formaldehyde are known by the United States Environment Protection Agency (EPA) to be carcinogenic chemicals for humans where exposure is via inhalation. Exposure to ethylene increases the risk of lymphoid cancer and, for females, breast cancer whilst formaldehyde causes myeloid leukemia and rare cancers, including cancers of the paranasal sinuses, nasal cavity, and nasopharynx.

The carbon dioxide generated from this process can speed up (or accelerate) the ripening of these fruits or vegetables where these fruits and vegetables are sensitive to both gases. Within the prior art some technologies have been established which seek to delay fruit and vegetable spoilage by addressing ethylene gas. However, such methods are ineffective in stopping spoilage, since as soon as ethylene gas is released from produce and exposed to oxygen, an oxidation process/reaction occurs, producing carbon dioxide. Because this oxidation process occurs instantly and continues to produce carbon dioxide, more rapidly than the technologies addressing the generation of ethylene can react. Accordingly, carbon dioxide is generated and current technologies cannot effectively slow down ripening or effectively reduce carbon dioxide emissions.

Accordingly, embodiments of the invention exploit a solution or powder of calcium hydroxide [Ca(OH)2] allowing carbon dioxide [CO2] to be absorbed from the atmosphere where fruits and vegetables are stored, and/or from packages etc. The solution of Ca(OH)2 reacts with CO2 as given by Equations (4A) and (4B). If excess CO2 in the storage room is added then a second reaction occurs (more CO2 can be absorbed) as given by Equations (5A) and (5B).

Calcium Hydroxide+Carbon Dioxide→Calcium Carbonate+Water (4A)

Ca(OH)₂+CO₂→CaCO₃+H₂O (4B)

Calcium Carbonate+Water+Carbon Dioxide→Calcium Bicarbonate (5A)

CaCO₃+H₂O+2CO₂→Ca(HCO₃)₂ (5B)

It is known that carbon dioxide increases produce spoilage and ripening. For example, farmers cut bananas from trees when they are green, and when they want them to mature, they place them in rooms full of carbon dioxide, to turn yellow in colour or control their environment by purging their storage area with nitrogen in order to delay ripening. Whilst supportable in bulk shipping/storage such systems are untenable at other points within the supply chain. Accordingly, there is a direct relationship between carbon dioxide and produce spoilage and ripening. Accordingly, it would be beneficial to provide farmers, consumers, retailers, suppliers and others within the overall life cycle of produce or products negatively impacted by carbon dioxide a low-cost technology that extends produce shelf life by reducing carbon dioxide from the produce's surrounding atmosphere.

Accordingly, in order to delay (or slow down) the acceleration and onset of ripening, the inventors have established a methodology of employing calcium hydroxide and or calcium carbonate in liquid solution and/or dried powder form to reduce carbon dioxide within a storage area or around the produce (e.g. fruit and vegetables) within shelving, displays areas etc. storage area, or from store shelves.

Further, some bacteria, which live on the surface of produce such as fruits and vegetables become more active in those areas of the surface that have access to carbon dioxide. Accordingly, by reducing the levels of carbon dioxide around produce the actions of these bacteria are slowed down further reducing the spoilage arising from these bacteria on the produce.

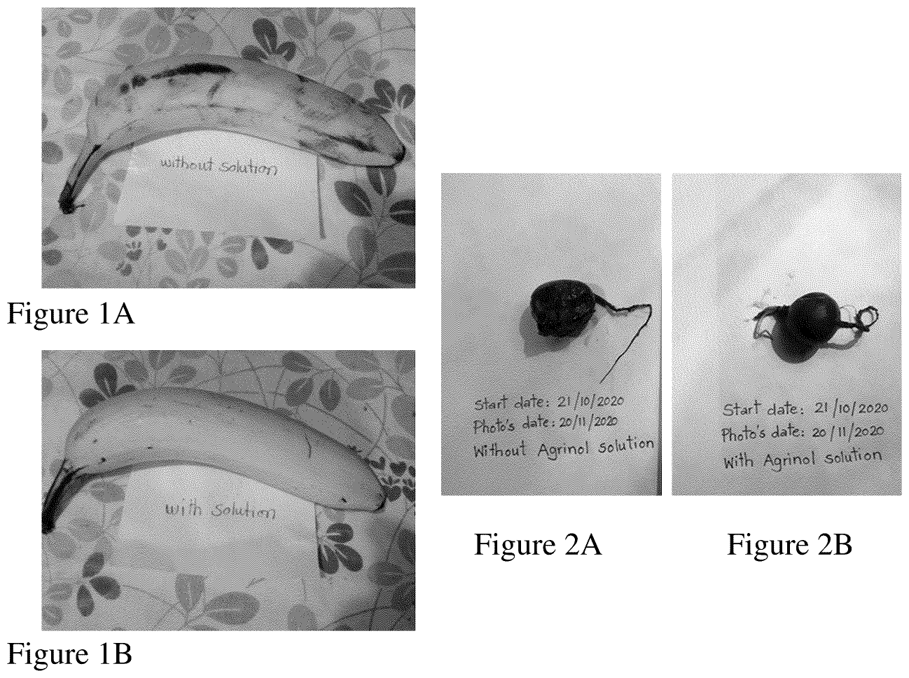

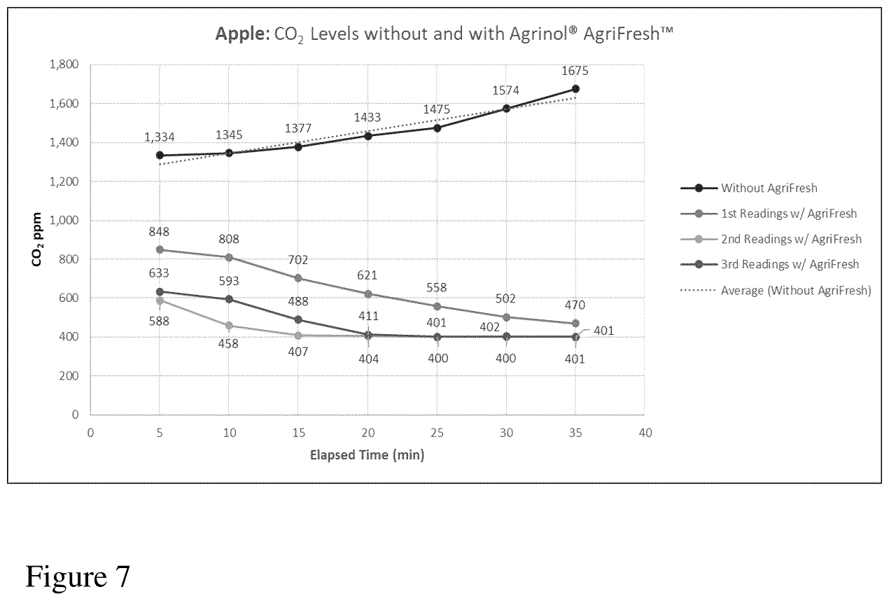

Accordingly, the inventors have established and conducted experiments to obtain data on a range of fruits and vegetables using an embodiment of the invention. Initial exemplary tests employing liquid or powder embodiments of the invention are presented below in FIGS. 1A to 6B. Subsequently, the inventors established tests presented below in FIGS. 7 to 20B respectively using an embodiment of the invention wherein a porous container (for example a pouch or bag) containing a food grade carbon dioxide absorbing powder (e.g. calcium hydroxide) was stored with the fruit and vegetables allowing the acquisition of data on the resulting levels of carbon dioxide relative to reference samples without embodiments of the invention. These porous containers within the storage container with the fruit allow air exchange and carbon dioxide absorption. The inventors employed a food grade powder within these tests such that rupture of the porous container would only release a food grade material reducing issues relating to contamination. However, it would be evident that within other embodiments of the invention that non-food grade chemicals may be employed where instructions to users to wash/clean produce upon rupture of the porous container would be provided.

As different produce release varying levels of ethylene gas then in some instances it would be beneficial for produce segregation to be employed discretely or in combination with embodiments of the invention. The objective being to prolong shelf-life and produce quality. Accordingly, produce (fruits or vegetables) that are sensitive to ethylene (C₂H₄) and carbon dioxide (CO₂) would not be mixed together in the same storage area or refrigeration shelf (refrigeration unit) etc.

Refrigeration and humidity control (e.g. high humidity) can help reduce the respiration of fruits and vegetable cells thereby effectively slowing down the metabolic processes within the produce's cells. This, in turn, reduces the rate of spoilage. Accordingly, whilst embodiments of the invention can be employed to reduce produce spoilage in open room temperature environments (e.g. retail outlet shelving etc.) they can also be employed in domestic and commercial refrigeration environments as well as during transportation and storage. It would be evident to one of skill in that art that embodiments of the invention by preserving produce in non-refrigerated environments results in reduced electricity consumption during the life-cycle of produce from harvesting to use, thereby further reducing carbon dioxide emissions from the produce life cycle and accordingly reducing the overall carbon footprint of produce storage and/or packaging with embodiments of the invention.

With respect to the generation of ethylene then examples of fruits and vegetables producing at high, medium, low, or very low levels are outlined below together with produce that does not generate ethylene. Accordingly, embodiments of the invention are particularly beneficial to those that produce ethylene but also reduce the spoilage of produce that does not generate ethylene by reducing the carbon dioxide within the environment surrounding the produce. This carbon dioxide arising, for example, transportation, relocation, storage, ambient environment and the natural human breathing of customers and staff in domestic and retail environments.
High Ethylene Generators: apples, peaches, pears, plums, apricots, avocados (ripe)
Medium Ethylene Generators: tomatoes (ripe), melons (honeydew), papaya, cantaloupe, guavas, mangoes, bananas (ripe)
Low Ethylene Generators: figs, kiwi (unripe), melons (crenshaw), precut fruit, tomatoes (mature green)
Very Low Ethylene Generators: cucumbers, grapefruit, lemons, oranges, strawberries
Produce not generating Ethylene: asparagus, artichokes, basil, beets, broccoli, cabbage, carrots, cauliflower, celery, coconuts, sweet corn, cranberries, eggplant, garlic, green beans, green peas, lettuce, mushrooms, onions, potatoes, watermelon

During initial experimentation, produce such as tomatoes, bananas, apples, cucumber, zucchinis, avocados, blueberries, radishes, lemons, parsley, and eggplants were tested by the inventors using a liquid solution application or using a housing with a permeable membrane casing containing a powder according to an embodiment of the invention within the container, where the housing is placed near the produce. However, it would be evident that the embodiments of the invention may be applied to fruit, vegetables, herbs and other undried organic produce.

Referring to FIGS. 1A and 1B there are depicted exemplary images of bananas showing reduced spoiling/ripening from application of a solution according to an embodiment of the invention. FIG. 1A depicts a banana stored as normal whilst FIG. 1B depicts another banana to which the liquid solution according to an embodiment of the invention was applied stored for the same period of time.

Referring to FIGS. 2A and 2B there are depicted exemplary images of beetroots showing reduced spoiling/ripening from application of a solution according to an embodiment of the invention. FIG. 2A depicts a beetroot stored as normal whilst FIG. 2B depicts another beetroot to which the liquid solution according to an embodiment of the invention was applied stored for the same period of time.

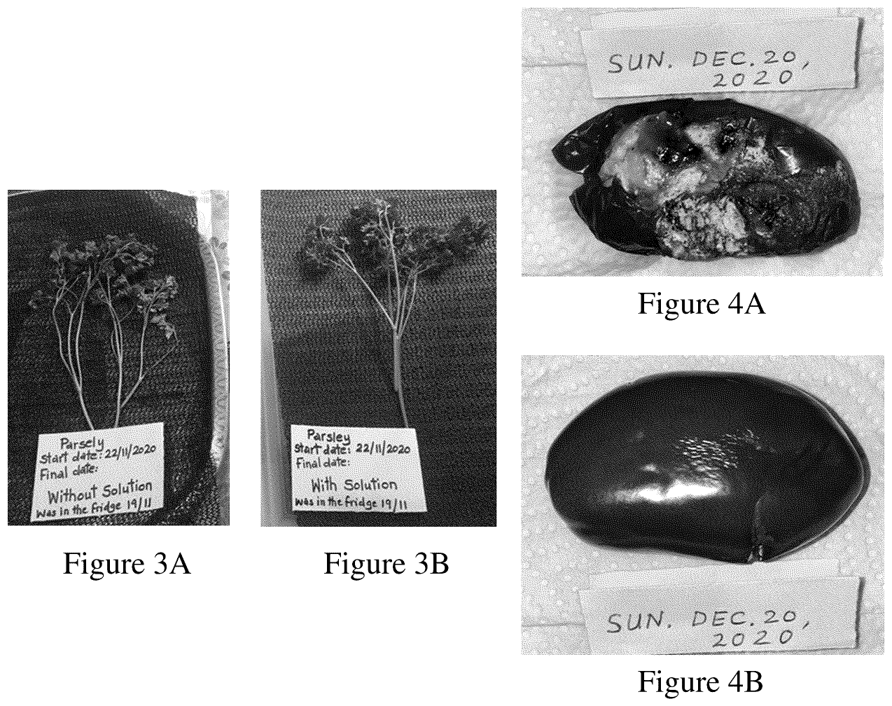

Referring to FIGS. 3A and 3B there are depicted exemplary images of parsley showing reduced spoiling/ripening from application of a solution according to an embodiment of the invention. FIG. 3A depicts some parsley stored as normal whilst FIG. 3B depicts other parsley to which the liquid solution according to an embodiment of the invention was applied stored for the same period of time.

Referring to FIGS. 4A and 4B there are depicted exemplary images of tomatoes showing reduced spoiling/ripening from application of a solution according to an embodiment of the invention. FIG. 4A depicts a tomato stored as normal whilst FIG. 4B depicts another tomato to which the liquid solution according to an embodiment of the invention was applied stored for the same period of time.

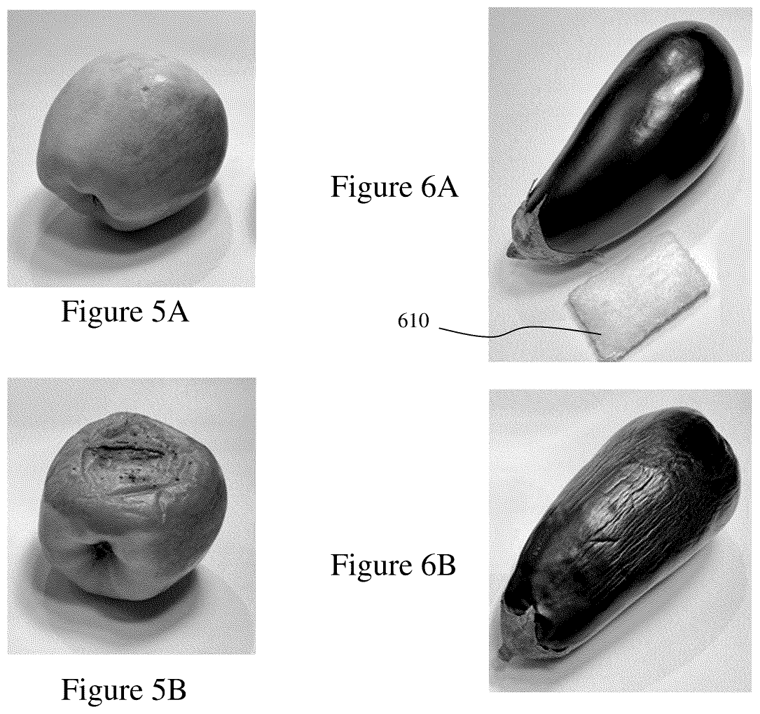

Referring to FIGS. 5A and 5B there are depicted exemplary images of apples showing reduced spoiling/ripening through an embodiment of the invention. FIG. 5A depicts an apple stored in a container with a housing, such as Housing 610 described below according to an embodiment of the invention. FIG. 5B depicts another apple stored for the same period of time in a container without the housing and without being treated with a solution according to an embodiment of the invention, such as the fruit and herbs depicted in FIGS. 1A, 2A, 3A, and 4A.

Accordingly, the apple in FIG. 5A is not spoilt and has been preserved in a condition in a comparable manner as the produce treated with a solution according to an embodiment of the invention as depicted in FIGS. 1B, 2B, 3B and 4B such that Housing 610 may also be employed discretely or in conjunction with treatment with a solution.

According to embodiments of the invention, the solution (comprising calcium hydroxide) may be applied directly to the produce where the solution is applied to the produce by at least one of dipping, spraying, misting, and coating.

Alternatively, the solution may be applied to a sheet which is then used to wrap around the produce wherein the solution may be applied to the sheet by at least one of dipping, spraying, misting, and coating.

Alternatively, the solution may be applied to one or more inner surfaces of a container within which produce is placed wherein the solution may be applied to the one or more inner surfaces of the container by at least one of dipping, spraying, misting, and coating.

Referring to FIGS. 6A and 6B there depicted exemplary images of aubergines (known in North America as eggplants) showing reduced spoiling/ripening through an embodiment of the invention. FIG. 6A depicts an aubergine which was stored within a container together with Housing 610, comprising a permeable membrane casing, containing a powder according to an embodiment of the invention powder such that the Housing 610 was in proximity to the aubergine. Housing 610 being referred to subsequently within this specification as a Housing 610, a Housing, or a Product Package. The aubergine being stored for the same period of time as the aubergine depicted in FIG. 6B. However, the aubergine in FIG. 6B was not treated with a liquid according to an embodiment of the invention, such as the fruit and herbs depicted in FIGS. 1A, 2A, 3A, and 4A or placed in proximity of a Housing 610 containing a material, e.g. powder, according to an embodiment of the invention.

Accordingly, the aubergine in FIG. 6A is not spoilt and has been preserved in a condition in a comparable manner as the produce treated with a solution according to an embodiment of the invention as depicted in FIGS. 1B, 2B, 3B and 4B such that Housing 610 may also be employed discretely or in conjunction with treatment with a solution.

Within the following descriptions with respect to FIGS. 7 to 20B exemplary volumes/quantities/weights of produce and absorbers according to embodiments of the invention are provided. However, it would be evident that these may be scaled to achieve related results, e.g. doubling the weight of the produce and the absorber. However, it would also be evident that the weight of produce may be scaled without adjusting the absorber thereby resulting in modified adjustments in results and accordingly resulting adjustments in produce spoilage.

In the following description, embodiments of the invention comprising the carbon dioxide absorber in food grade powder form within porous container (pouch, bag, etc.) are hereinafter referred to as a Housing or Packaged Product, such as Housing 610 for example as marketed under the brand name AgriFresh™ by the inventors.

### Experiment A: Apples

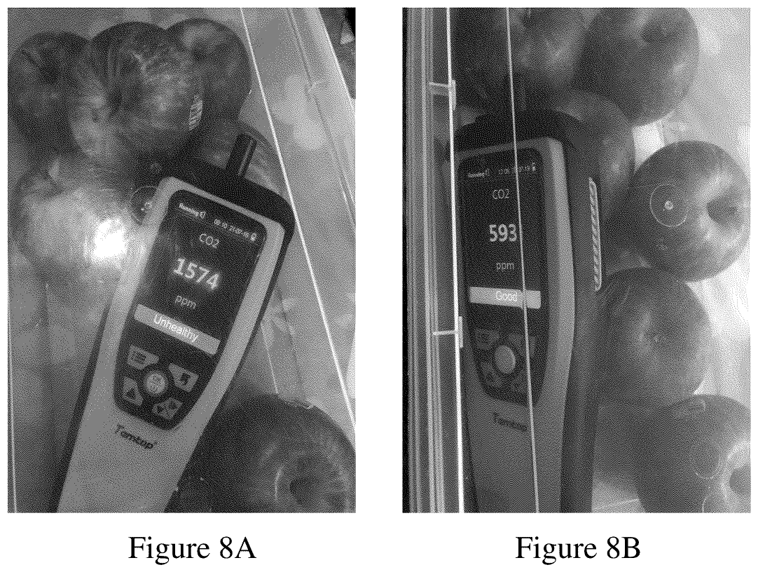

Referring to FIG. 7 there is depicted a graph of the short term evolution of carbon dioxide levels of packaged apples with and without a Product Package according to an embodiment of the invention. FIGS. 8A and 8B depict packaged apples with and without a Product Package according to an embodiment of the invention with a sensor device depicting the carbon dioxide levels.

Produce
Quantity=6
Total Weight=1,062 grams
Retail Box Size=30 cm×17 cm×11 cm

Table 1 below presents the readings of carbon dioxide without an embodiment of the invention within the Retail Box. 7 measurements were taken, each 5 minutes apart, wherein the first reading (i.e. at 5 minutes elapsed) was ignored to allow for equipment calibration and stability.

TABLE 1
Carbon Dioxide versus Time for Apples
without Embodiment of the Invention

| Reading No. | Elapsed Time (min) | CO₂ Concentration (ppm) |
|---|---|---|
| 1 | 5 | 1,334 |
| 2 | 10 | 1,345 |
| 3 | 15 | 1,377 |
| 4 | 20 | 1,433 |
| 5 | 25 | 1,475 |
| 6 | 30 | 1,574 |
| 7 | 35 | 1,675 |

Average CO₂ Concentration (ppm) = 1,480

Accordingly, the average CO2 concentration naturally produced by 1,062 grams of apples was 1,480 ppm in the 30 minutes. Accordingly, the short-term average CO2 concentration naturally produced by apples is 1,393 ppm/kg.

Table 2 below presents the readings of carbon dioxide with 2 Product Packages placed within the vicinity of the produce after the apples and Product Packages had been in the container for 24 hours. Each Product Package containing 4 grams of powder. Again the initial reading was ignored.

TABLE 2
Carbon Dioxide versus Time for Apples with
Embodiment of the Invention after 24 Hours

| Reading No. | Elapsed Time (min) | CO₂ Concentration (ppm) |
|---|---|---|
| 1 | 5 | 848 |
| 2 | 10 | 808 |
| 3 | 15 | 702 |
| 4 | 20 | 621 |
| 5 | 25 | 558 |
| 6 | 30 | 502 |
| 7 | 35 | 470 |

Average CO₂ Concentration (ppm) = 610

Accordingly, the average CO2 concentration naturally produced by 1,062 grams of apples with the pair of Product Packages was 610 ppm in the 30 minutes. Accordingly, the short-term average CO2 concentration in the vicinity of the apples with the Product Packages was 575 ppm/kg.

Repeating the measurements after 48 and 72 hours yielded the results in Tables 3 and 4 below. Accordingly, after 48 hours the carbon dioxide concentration from a nominal 1 kg of apples was reduced to 388 ppm and after 72 hours to 423 ppm.

TABLE 3
Carbon Dioxide versus Time for Apples with
Embodiment of the Invention after 48 Hours

| Reading No. | Elapsed Time (min) | CO₂ Concentration (ppm) |
|---|---|---|
| 1 | 5 | 588 |
| 2 | 10 | 458 |
| 3 | 15 | 407 |
| 4 | 20 | 404 |
| 5 | 25 | 400 |
| 6 | 30 | 400 |
| 7 | 35 | 401 |

Average CO₂ Concentration (ppm) = 412

TABLE 4
Carbon Dioxide versus Time for Apples with
Embodiment of the Invention after 72 Hours

| Reading No. | Elapsed Time (min) | CO₂ Concentration (ppm) |
|---|---|---|
| 1 | 5 | 633 |
| 2 | 10 | 593 |
| 3 | 15 | 488 |
| 4 | 20 | 411 |
| 5 | 25 | 401 |
| 6 | 30 | 402 |
| 7 | 35 | 401 |

Average CO₂ Concentration (ppm) = 449

Accordingly, without Product Packages, the average CO2 concentration naturally produced by 1,000 grams of apples (i.e. on its own) was found to be 1,393 ppm. With Product Packages absorbing continuously for 72 hours, the average CO2 concentration of each of the 3 consecutive days, for 1,000 grams of apples, was found to be:
1st day (0 h to 24 h)=575 ppm CO2
2nd day (24 h to 48 h)=388 ppm CO2
3rd day (48 h to 72 h)=423 ppm CO2

Hence, for a 1000-gram batch of apples over an elapsed duration of 72 hours (i.e. the same bags running continuously over 3 consecutive days), the inventive Product Packages reduced the average CO2 concentration from 1,393 ppm CO2 to 575 ppm CO2 in 1st day (0 h to 24 h), 388 ppm CO2 in 2nd day (24 h to 48 h), and 423 ppm CO2 in 3rd day (48 h to 72 h). Accordingly, the 2 Product Packages with 4 grams of absorber within each absorbed 59% of CO2 gases in 1st day, 72% in 2nd day, and 70% in 3rd day.

### Experiment B: Red Peppers

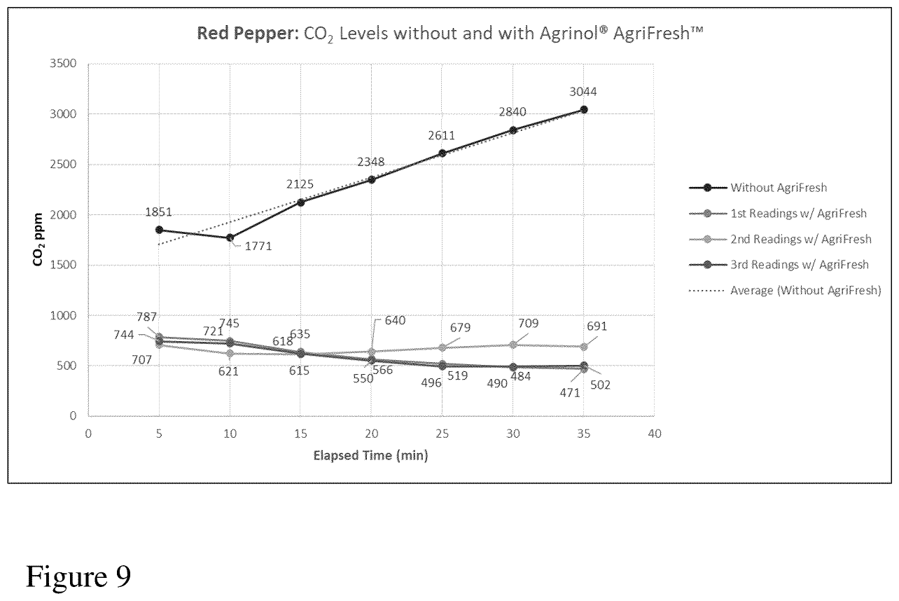

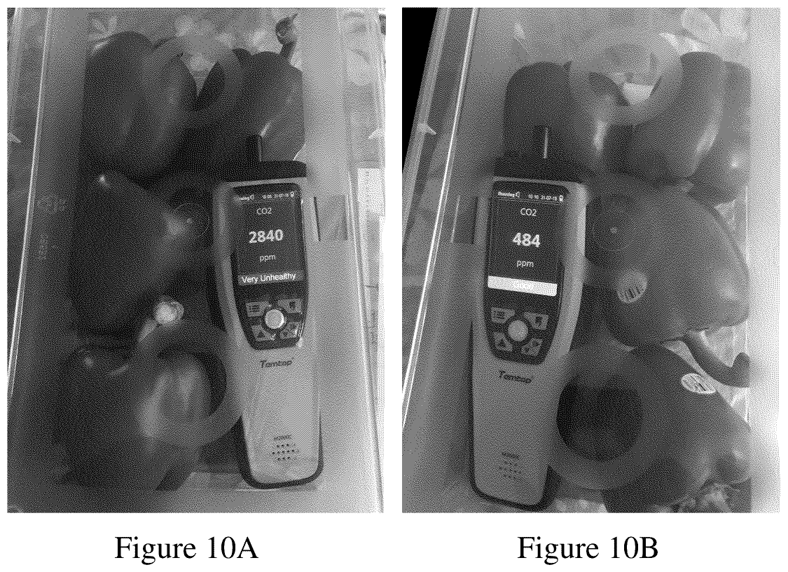

Referring to FIG. 9 there is depicted a graph of the short term evolution of carbon dioxide levels of packaged red peppers with and without a Product Package according to an embodiment of the invention. FIGS. 10A and 10B depict packaged red peppers with and without a Product Package according to an embodiment of the invention with a sensor device depicting the carbon dioxide levels.

Produce
Quantity=4
Total Weight=867 grams
Retail Box Size=30 cm×17 cm×11 cm

Table 5 below presents the readings of carbon dioxide without an embodiment of the invention within the Retail Box. 7 measurements were taken, each 5 minutes apart, wherein the first reading (i.e. at 5 minutes elapsed) was ignored to allow for equipment calibration and stability.

TABLE 5
Carbon Dioxide versus Time for Red Peppers
without Embodiment of the Invention

| Reading No. | Elapsed Time (min) | CO₂ Concentration (ppm) |
|---|---|---|
| 1 | 5 | 1,851 |
| 2 | 10 | 1,772 |
| 3 | 15 | 2,125 |
| 4 | 20 | 2,348 |
| 5 | 25 | 2,611 |
| 6 | 30 | 2,840 |
| 7 | 35 | 3,044 |

Average CO₂ Concentration (ppm) = 2,457

Accordingly, the average CO2 concentration naturally produced by 867 grams of red peppers was 2,457 ppm in the 30 minutes. Accordingly, the short-term average CO2 concentration naturally produced by apples is 2,833 ppm/kg.

Table 6 below presents the readings of carbon dioxide with 3 Product Packages bags placed within the vicinity of the produce after the red peppers and Product Packages had been in the container for 24 hours. Each Product Package containing 4 grams of powder. Again the initial reading was ignored.

TABLE 6
Carbon Dioxide versus Time for Red Peppers with
Embodiment of the Invention after 24 Hours

| Reading No. | Elapsed Time (min) | CO₂ Concentration (ppm) |
|---|---|---|
| 1 | 5 | 787 |
| 2 | 10 | 745 |
| 3 | 15 | 635 |
| 4 | 20 | 566 |
| 5 | 25 | 519 |
| 6 | 30 | 484 |
| 7 | 35 | 471 |

Average CO₂ Concentration (ppm) = 570

Accordingly, the average CO2 concentration naturally produced by 867 grams of red peppers with the pair of Product Packages was 570 ppm in the 30 minutes. Accordingly, the short-term average CO2 concentration in the vicinity of the red peppers with the Product Packages was 657 ppm/kg.

Repeating the measurements after 48 and 72 hours yielded the results in Tables 7 and 8 below. Accordingly, after 48 hours the carbon dioxide concentration from a nominal 1 kg of red peppers was reduced to 760 ppm and after 72 hours to 649 ppm.

TABLE 7
Carbon Dioxide versus Time for Red Peppers with
Embodiment of the Invention after 48 Hours

| Reading No. | Elapsed Time (min) | CO₂ Concentration (ppm) |
|---|---|---|
| 1 | 5 | 707 |
| 2 | 10 | 621 |
| 3 | 15 | 615 |
| 4 | 20 | 640 |
| 5 | 25 | 679 |
| 6 | 30 | 709 |
| 7 | 35 | 691 |

Average CO₂ Concentration (ppm) = 659

TABLE 8
Carbon Dioxide versus Time for Red Peppers with
Embodiment of the Invention after 72 Hours

| Reading No. | Elapsed Time (min) | CO₂ Concentration (ppm) |
|---|---|---|
| 1 | 5 | 744 |
| 2 | 10 | 721 |
| 3 | 15 | 618 |
| 4 | 20 | 550 |
| 5 | 25 | 496 |
| 6 | 30 | 490 |
| 7 | 35 | 502 |

Average CO₂ Concentration (ppm) = 563

Accordingly, without Product Packages, the average CO2 concentration naturally produced by 1,000 grams of red peppers (i.e. on its own) was found to be 2,833 ppm. With Product Packages absorbing continuously for 72 hours, the average CO2 concentration of each of the 3 consecutive days, for 1,000 grams of red pepper, was found to be:
1st day (0 h to 24 h)=657 ppm CO2
2nd day (24 h to 48 h)=760 ppm CO2
3rd day (48 h to 72 h)=649 ppm CO2

Hence, for a 1000-gram batch of red peppers over an elapsed duration of 72 hours (i.e. the same bags running continuously over 3 consecutive days), the inventive Product Packages reduced the average CO2 concentration from 2,833 ppm CO2 to 657 ppm CO2 in 1st day (0 h to 24 h), 760 ppm CO2 in 2nd day (24 h to 48 h), and 649 ppm CO2 in 3rd day (48 h to 72 h). Accordingly, the 3 Product Packages with 4 grams of absorber within each absorbed 77% of CO2 gases in 1st day, 73% in 2nd day, and 77% in 3rd day.

### Experiment C: Red Potatoes

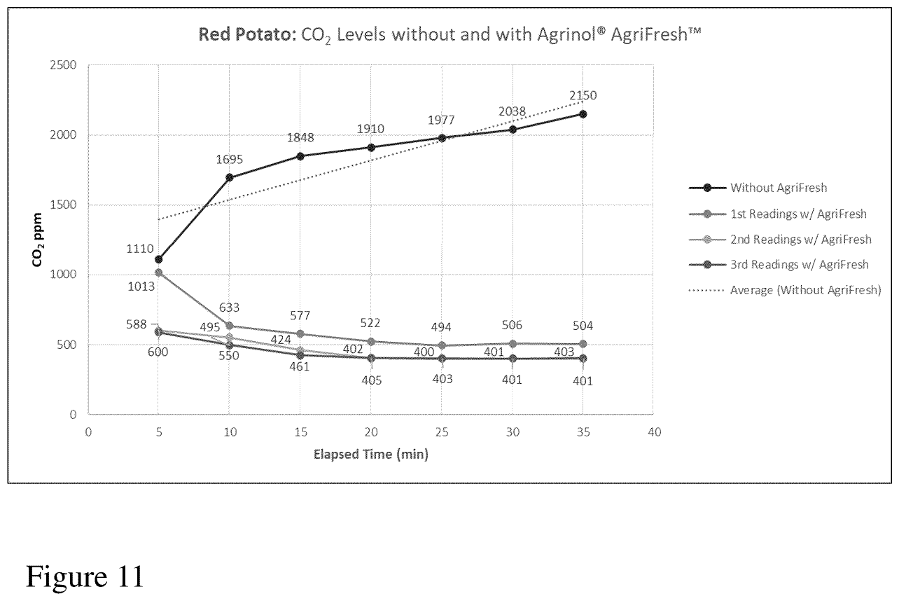

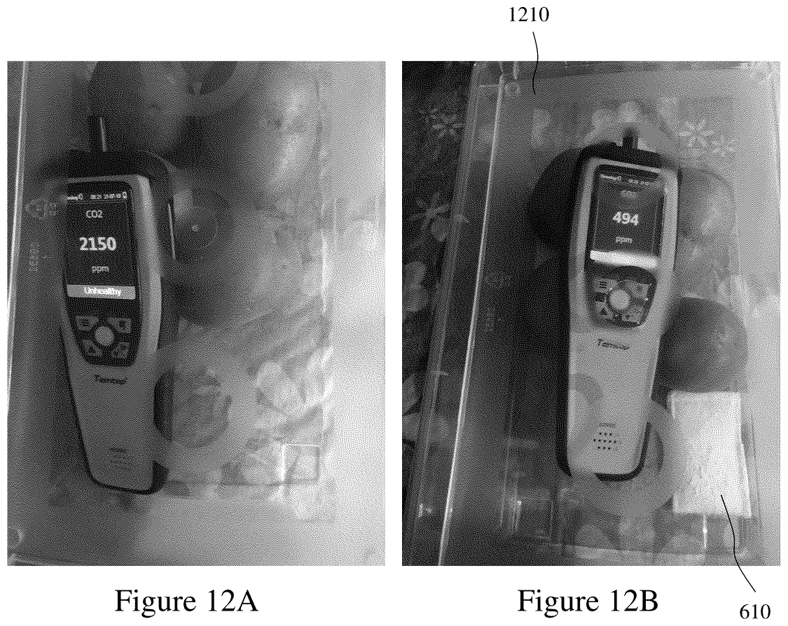

Referring to FIG. 11 there is depicted a graph of the short term evolution of carbon dioxide levels of packaged red potatoes with and without a Product Package according to an embodiment of the invention. FIGS. 12A and 12B depict packaged red potatoes with and without a Product Package according to an embodiment of the invention with a sensor device depicting the carbon dioxide levels. A Housing 610 being visible within the Container 1210 within which the Housings 610 (as a pair were employed), the red potatoes and the CO2 Meter were placed.

Produce
Quantity=4
Total Weight=887 grams
Retail Box Size=30 cm×17 cm×11 cm

Table 9 below presents the readings of carbon dioxide without an embodiment of the invention within the Retail Box. 7 measurements were taken, each 5 minutes apart, wherein the first reading (i.e. at 5 minutes elapsed) was ignored to allow for equipment calibration and stability.

TABLE 9
Carbon Dioxide versus Time for Red Potatoes
without Embodiment of the Invention

| Reading No. | Elapsed Time (min) | CO₂ Concentration (ppm) |
|---|---|---|
| 1 | 5 | 1,110 |
| 2 | 10 | 1,695 |
| 3 | 15 | 1,848 |
| 4 | 20 | 1,910 |
| 5 | 25 | 1,977 |
| 6 | 30 | 2,038 |
| 7 | 35 | 2.150 |

Average CO₂ Concentration (ppm) = 1,936

Accordingly, the average CO2 concentration naturally produced by 887 grams of red potatoes was 1,936 ppm in the 30 minutes. Accordingly, the short-term average CO2 concentration naturally produced by is 2,183 ppm/kg.

Table 10 below presents the readings of carbon dioxide with 2 Product Packages bags placed within the vicinity of the produce after the red potatoes and Product Packages had been in the container for 24 hours. Each Product Package containing 4 grams of powder. Again the initial reading was ignored.

TABLE 10
Carbon Dioxide versus Time for Red Potatoes with
Embodiment of the Invention after 24 Hours

| Reading No. | Elapsed Time (min) | CO₂ Concentration (ppm) |
|---|---|---|
| 1 | 5 | 1,013 |
| 2 | 10 | 633 |
| 3 | 15 | 577 |
| 4 | 20 | 522 |
| 5 | 25 | 494 |
| 6 | 30 | 506 |
| 7 | 35 | 504 |

Average CO₂ Concentration (ppm) = 539

Accordingly, the average CO2 concentration naturally produced by 887 grams of red potatoes with the pair of Product Packages was 539 ppm in the 30 minutes. Accordingly, the short-term average CO2 concentration in the vicinity of the red potatoes with the Product Packages was 608 ppm/kg.

Repeating the measurements after 48 and 72 hours yielded the results in Tables 11 and 12 below. Accordingly, after 48 hours the carbon dioxide concentration from a nominal 1 kg of red potatoes was reduced to 492 ppm and after 72 hours to 474 ppm.

TABLE 11
Carbon Dioxide versus Time for Red Potatoes with
Embodiment of the Invention after 48 Hours

| Reading No. | Elapsed Time (min) | CO₂ Concentration (ppm) |
|---|---|---|
| 1 | 5 | 600 |
| 2 | 10 | 550 |
| 3 | 15 | 461 |
| 4 | 20 | 405 |
| 5 | 25 | 403 |
| 6 | 30 | 401 |
| 7 | 35 | 401 |

Average CO₂ Concentration (ppm) = 437

TABLE 12
Carbon Dioxide versus Time for Red Potatoes with
Embodiment of the Invention after 72 Hours

| Reading No. | Elapsed Time (min) | CO₂ Concentration (ppm) |
|---|---|---|
| 1 | 5 | 588 |
| 2 | 10 | 495 |
| 3 | 15 | 424 |
| 4 | 20 | 402 |
| 5 | 25 | 400 |
| 6 | 30 | 401 |
| 7 | 35 | 403 |

Average CO₂ Concentration (ppm) = 421

Accordingly, without Product Packages, the average CO2 concentration naturally produced by 1,000 grams of red potatoes (i.e. on its own) was found to be 2,183 ppm. With Product Packages absorbing continuously for 72 hours, the average CO2 concentration of each of the 3 consecutive days, for 1,000 grams of red potatoes was found to be:
1st day (0 h to 24 h)=608 ppm CO2
2nd day (24 h to 48 h)=492 ppm CO2
3rd day (48 h to 72 h)=474 ppm CO2

Hence, for a 1000-gram batch of red potatoes over an elapsed duration of 72 hours (i.e. the same bags running continuously over 3 consecutive days), the inventive Product Packages reduced the average CO2 concentration from ppm CO2 to 608 ppm CO2 in 1st day (0 h to 24 h), 492 ppm CO2 in 2nd day (24 h to 48 h), and 474 ppm CO2 in 3rd day (48 h to 72 h). Accordingly, the 2 Product Packages with 4 grams of absorber within each absorbed 72% of CO2 gases in 1st day, 77% in 2nd day, and 78% in 3rd day.

### Experiment D: Red Onions

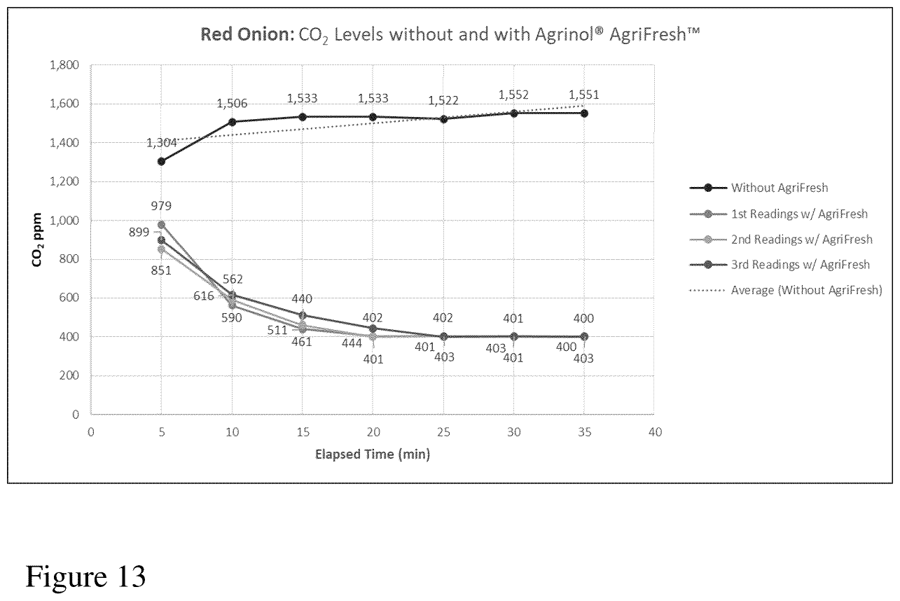

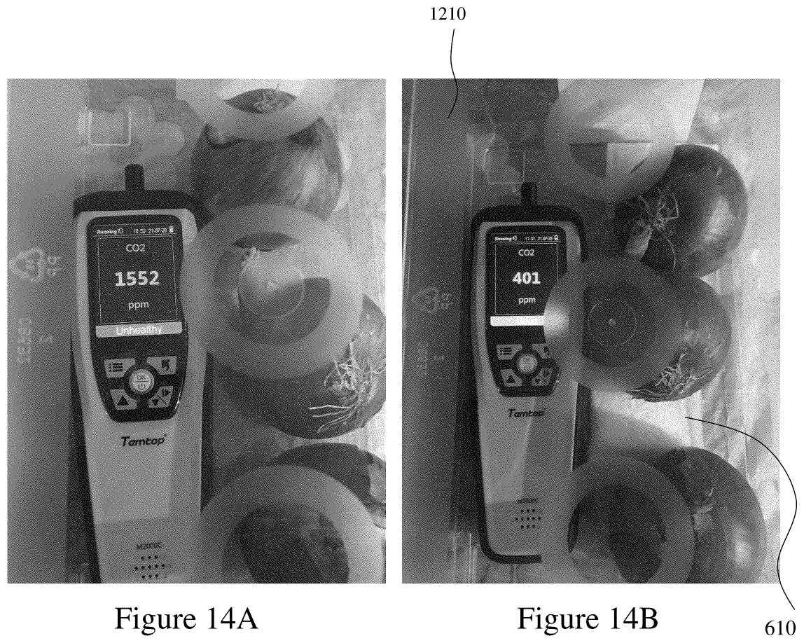

Referring to FIG. 13 there is depicted a graph of the short term evolution of carbon dioxide levels of packaged red onion with and without a Product Package according to an embodiment of the invention. FIGS. 14A and 14B depict packaged red onion with and without a Product Package according to an embodiment of the invention with a sensor device depicting the carbon dioxide levels. Housings 610 being visible within the Container 1210 within which the Housings 610, the red onions and the CO2 Meter were placed.

Produce
Quantity=3
Total Weight=633 grams
Retail Box Size=30 cm×17 cm×11 cm

Table 13 below presents the readings of carbon dioxide without an embodiment of the invention within the Retail Box. 7 measurements were taken, each 5 minutes apart, wherein the first reading (i.e. at 5 minutes elapsed) was ignored to allow for equipment calibration and stability.

TABLE 13
Carbon Dioxide versus Time for Red Onions
without Embodiment of the Invention

| Reading No. | Elapsed Time (min) | CO₂ Concentration (ppm) |
|---|---|---|
| 1 | 5 | 1,304 |
| 2 | 10 | 1,506 |
| 3 | 15 | 1,533 |
| 4 | 20 | 1,533 |
| 5 | 25 | 1,522 |
| 6 | 30 | 1,552 |
| 7 | 35 | 1,551 |

Average CO₂ Concentration (ppm) = 1,533

Accordingly, the average CO2 concentration naturally produced by 1,533 grams of was ppm in the 30 minutes. Accordingly, the short-term average CO2 concentration naturally produced by is 2,422 ppm/kg.

Table 14 below presents the readings of carbon dioxide with 2 Product Packages placed within the vicinity of the produce after the red onions and Product Packages had been in the container for 24 hours. Each Product Package containing 4 grams of powder. Again the initial reading was ignored.

TABLE 14
Carbon Dioxide versus Time for Red Onions with
Embodiment of the Invention after 24 Hours

| Reading No. | Elapsed Time (min) | CO₂ Concentration (ppm) |
|---|---|---|
| 1 | 5 | 979 |
| 2 | 10 | 562 |
| 3 | 15 | 440 |
| 4 | 20 | 402 |
| 5 | 25 | 402 |
| 6 | 30 | 401 |
| 7 | 35 | 400 |

Average CO₂ Concentration (ppm) = 435

Accordingly, the average CO2 concentration naturally produced by 663 grams of red onions with the pair of Product Packages was 435 ppm in the 30 minutes. Accordingly, the short-term average CO2 concentration in the vicinity of the red onions with the Product Packages was 686 ppm/kg.

Repeating the measurements after 48 and 72 hours yielded the results in Tables 15 and 16 below. Accordingly, after 48 hours the carbon dioxide concentration from a nominal 1 kg of red onions was reduced to 700 ppm and after 72 hours to 731 ppm.

TABLE 15
Carbon Dioxide versus Time for Red Onions with
Embodiment of the Invention after 48 Hours

| Reading No. | Elapsed Time (min) | CO₂ Concentration (ppm) |
|---|---|---|
| 1 | 5 | 851 |
| 2 | 10 | 590 |
| 3 | 15 | 461 |
| 4 | 20 | 401 |
| 5 | 25 | 403 |
| 6 | 30 | 401 |
| 7 | 35 | 403 |

Average CO₂ Concentration (ppm) = 443

TABLE 16
Carbon Dioxide versus Time for Red Onions with
Embodiment of the Invention after 72 Hours

| Reading No. | Elapsed Time (min) | CO₂ Concentration (ppm) |
|---|---|---|
| 1 | 5 | 899 |
| 2 | 10 | 616 |
| 3 | 15 | 511 |
| 4 | 20 | 444 |
| 5 | 25 | 401 |
| 6 | 30 | 403 |
| 7 | 35 | 400 |

Average CO₂ Concentration (ppm) = 463

Accordingly, without Product Packages, the average CO2 concentration naturally produced by 1,000 grams of red onions (i.e. on its own) was found to be 2,422 ppm. With Product Packages absorbing continuously for 72 hours, the average CO2 concentration of each of the 3 consecutive days, for 1,000 grams of red onions, was found to be:
1st day (0 h to 24 h)=686 ppm CO2
2nd day (24 h to 48 h)=700 ppm CO2
3rd day (48 h to 72 h)=731 ppm CO2

Hence, for a 1000-gram batch of red onions over an elapsed duration of 72 hours (i.e. the same bags running continuously over 3 consecutive days), the inventive Product Packages reduced the average CO2 concentration from 2,422 ppm CO2 to 686 ppm CO2 in 1st day (0 h to 24 h), 700 ppm CO2 in 2nd day (24 h to 48 h), and 731 ppm CO2 in 3rd day (48 h to 72 h). Accordingly, the 2 Product Packages with 4 grams of absorber within each absorbed 72% of CO2 gases in 1st day, 71% in 2nd day, and 70% in 3rd day.

### Experiment E: Yellow Onions

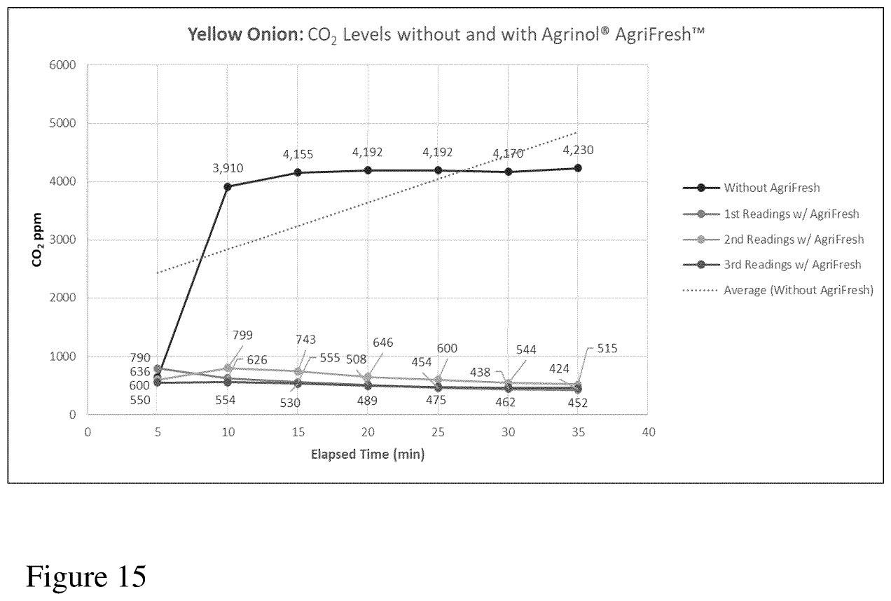

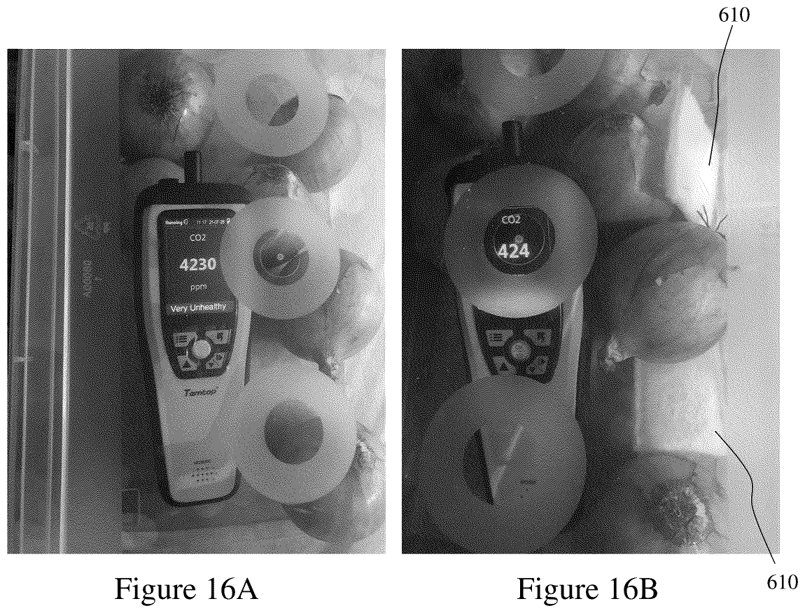

Referring to FIG. 15 there is depicted a graph of the short term evolution of carbon dioxide levels of packaged yellow onions with and without a Product Package according to an embodiment of the invention. FIGS. 16A and 16B depict packaged yellow onions with and without a Product Package according to an embodiment of the invention with a sensor device depicting the carbon dioxide levels. Housings 610 being visible within a container within which the Housings 610, the yellow onions and the CO2 Meter were placed.

Produce
Quantity=6
Total Weight=1,045 grams
Retail Box Size=33 cm×20 cm×13 cm

Table 17 below presents the readings of carbon dioxide without an embodiment of the invention within the Retail Box. 7 measurements were taken, each 5 minutes apart, wherein the first reading (i.e. at 5 minutes elapsed) was ignored to allow for equipment calibration and stability.

TABLE 17
Carbon Dioxide versus Time for Yellow Onions
without Embodiment of the Invention

| Reading No. | Elapsed Time (min) | CO₂ Concentration (ppm) |
|---|---|---|
| 1 | 5 | 636 |
| 2 | 10 | 3,910 |
| 3 | 15 | 4,155 |
| 4 | 20 | 4,192 |
| 5 | 25 | 4,192 |
| 6 | 30 | 4,170 |
| 7 | 35 | 4,230 |

Average CO₂ Concentration (ppm) = 4,142

Accordingly, the average CO2 concentration naturally produced by 1,045 grams of yellow onions was 4,142 ppm in the 30 minutes. Accordingly, the short-term average CO2 concentration naturally produced by is 3,963 ppm/kg.

Table 18 below presents the readings of carbon dioxide with 2 Product Packages placed within the vicinity of the produce after the yellow onions and Product Packages had been in the container for 24 hours. Each Product Package containing 4 grams of powder. Again the initial reading was ignored.

TABLE 18
Carbon Dioxide versus Time for Yellow Onions with
Embodiment of the Invention after 24 Hours

| Reading No. | Elapsed Time (min) | CO₂ Concentration (ppm) |
|---|---|---|
| 1 | 5 | 790 |
| 2 | 10 | 626 |
| 3 | 15 | 555 |
| 4 | 20 | 508 |
| 5 | 25 | 454 |
| 6 | 30 | 438 |
| 7 | 35 | 424 |

Average CO₂ Concentration (ppm) = 501

Accordingly, the average CO2 concentration naturally produced by grams of 1,045 with the pair of Product Packages was 501 ppm in the 30 minutes. Accordingly, the short-term average CO2 concentration in the vicinity of the yellow onions with the Product Packages bags was 479 ppm/kg.

Repeating the measurements after 48 and 72 hours yielded the results in Tables 19 and 20 below. Accordingly, after 48 hours the carbon dioxide concentration from a nominal 1 kg of yellow onions was reduced to 614 ppm and after 72 hours to 472 ppm.

TABLE 19
Carbon Dioxide versus Time for Yellow Onions with
Embodiment of the Invention after 48 Hours

| Reading No. | Elapsed Time (min) | CO₂ Concentration (ppm) |
|---|---|---|
| 1 | 5 | 600 |
| 2 | 10 | 799 |
| 3 | 15 | 743 |
| 4 | 20 | 646 |
| 5 | 25 | 600 |
| 6 | 30 | 544 |
| 7 | 35 | 515 |

Average CO₂ Concentration (ppm) = 641

TABLE 20
Carbon Dioxide versus Time for Yellow Onions with
Embodiment of the Invention after 72 Hours

| Reading No. | Elapsed Time (min) | CO₂ Concentration (ppm) |
|---|---|---|
| 1 | 5 | 550 |
| 2 | 10 | 554 |
| 3 | 15 | 530 |
| 4 | 20 | 489 |
| 5 | 25 | 475 |
| 6 | 30 | 462 |
| 7 | 35 | 452 |

Average CO₂ Concentration (ppm) = 494

Accordingly, without Product Packages, the average CO2 concentration naturally produced by 1,000 grams of yellow onions (i.e. on its own) was found to be 3,963 ppm. With Product Packages absorbing continuously for 72 hours, the average CO2 concentration of each of the 3 consecutive days, for 1,000 grams of yellow onions, was found to be:
1st day (0 h to 24 h)=479 ppm CO2
2nd day (24 h to 48 h)=614 ppm CO2
3rd day (48 h to 72 h)=472 ppm CO2

Hence, for a 1000-gram batch of yellow onions over an elapsed duration of 72 hours (i.e. the same bags running continuously over 3 consecutive days), the inventive Product Packages reduced the average CO2 concentration from 3,963 ppm CO2 to 479 ppm CO2 in 1st day (0 h to 24 h), 614 ppm CO2 in 2nd day (24 h to 48 h), and 472 ppm CO2 in 3rd day (48 h to 72 h). Accordingly, the 2 Product Packages with 4 grams of absorber within each absorbed 88% of CO2 gases in 1st day, 85% in 2nd day, and 88% in 3rd day.

### Experiment F: Garlic

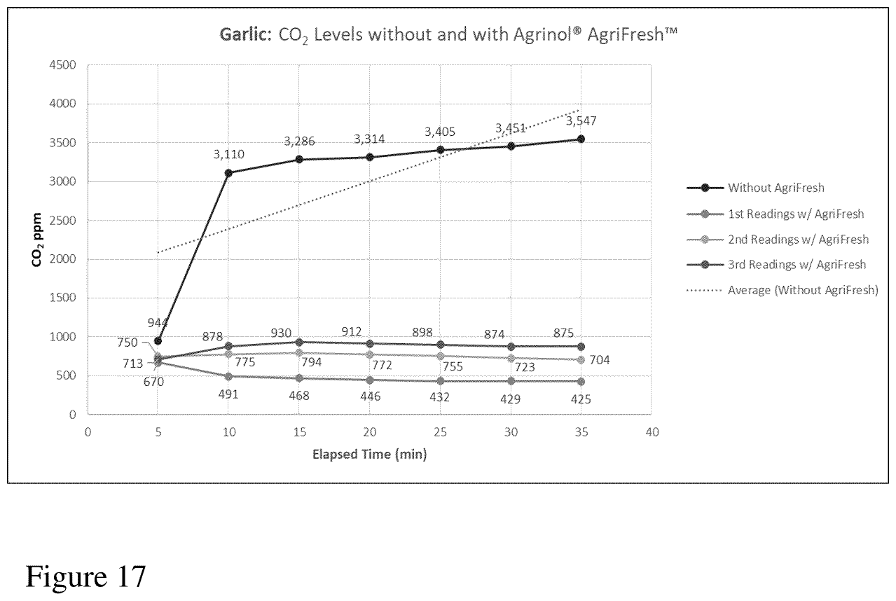

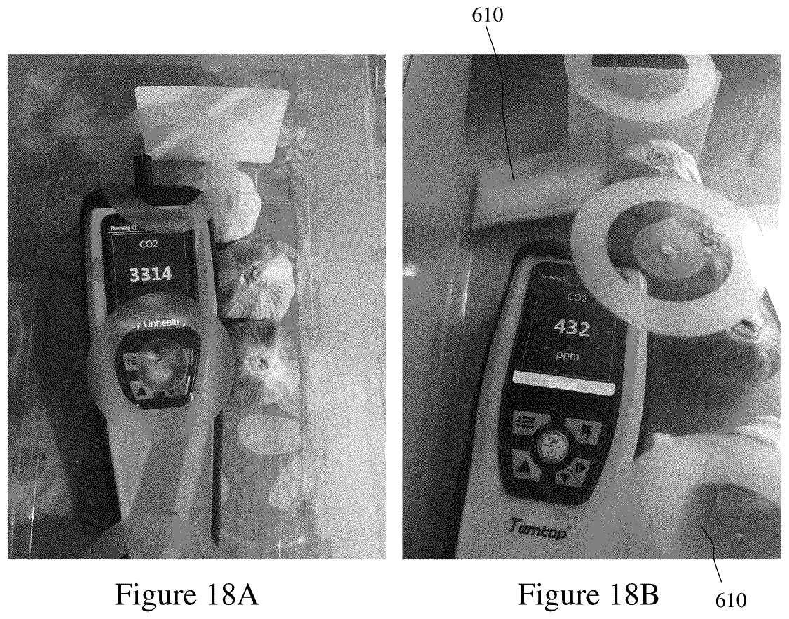

Referring to FIG. 17 there is depicted a graph of the short term evolution of carbon dioxide levels of packaged garlic with and without a Product Package according to an embodiment of the invention. FIGS. 18A and 18B depict packaged garlic with and without a Product Package according to an embodiment of the invention with a sensor device depicting the carbon dioxide levels. A Housing 610 being visible within a container within which the Housings 610, the garlic and the CO2 Meter were placed.

Produce
Quantity=4 heads
Total Weight=174 grams
Retail Box Size=30 cm×17 cm×11 cm

Table 21 below presents the readings of carbon dioxide without an embodiment of the invention within the Retail Box. 7 measurements were taken, each 5 minutes apart, wherein the first reading (i.e. at 5 minutes elapsed) was ignored to allow for equipment calibration and stability.

TABLE 21
Carbon Dioxide versus Time for Garlic
without Embodiment of the Invention

| Reading No. | Elapsed Time (min) | CO₂ Concentration (ppm) |
|---|---|---|
| 1 | 5 | 944 |
| 2 | 10 | 3,110 |
| 3 | 15 | 3,286 |
| 4 | 20 | 3,314 |
| 5 | 25 | 3,405 |
| 6 | 30 | 3,451 |
| 7 | 35 | 3,547 |

Average CO₂ Concentration (ppm) = 3,352

Accordingly, the average CO2 concentration naturally produced by 174 grams of garlic was 3,352 ppm in the 30 minutes. Accordingly, the short-term average CO2 concentration naturally produced by is 19,265 ppm/kg.

Table 22 below presents the readings of carbon dioxide with 2 Product Packages placed within the vicinity of the produce after the garlic and Product Packages had been in the container for 24 hours. Each Product Package containing 4 grams of powder. Again the initial reading was ignored.

TABLE 22
Carbon Dioxide versus Time for Garlic with
Embodiment of the Invention after 24 Hours

| Reading No. | Elapsed Time (min) | CO₂ Concentration (ppm) |
|---|---|---|
| 1 | 5 | 670 |
| 2 | 10 | 491 |
| 3 | 15 | 468 |
| 4 | 20 | 446 |
| 5 | 25 | 432 |
| 6 | 30 | 429 |
| 7 | 35 | 425 |

Average CO₂ Concentration (ppm) = 449

Accordingly, the average CO2 concentration naturally produced by 174 grams of garlic with the pair of Product Packages was 449 ppm in the 30 minutes. Accordingly, the short-term average CO2 concentration in the vicinity of the garlic with the Product Packages was 2,578 ppm/kg.

Repeating the measurements after 48 and 72 hours yielded the results in Tables 23 and 24 below. Accordingly, after 48 hours the carbon dioxide concentration from a nominal 1 kg of garlic was reduced to 4,332 ppm and after 72 hours to 5,141 ppm.

TABLE 23
Carbon Dioxide versus Time for Garlic with
Embodiment of the Invention after 48 Hours

| Reading No. | Elapsed Time (min) | CO₂ Concentration (ppm) |
|---|---|---|
| 1 | 5 | 750 |
| 2 | 10 | 775 |
| 3 | 15 | 794 |
| 4 | 20 | 772 |
| 5 | 25 | 755 |
| 6 | 30 | 723 |
| 7 | 35 | 704 |

Average CO₂ Concentration (ppm) = 754

TABLE 24
Carbon Dioxide versus Time for Garlic with
Embodiment of the Invention after 72 Hours

| Reading No. | Elapsed Time (min) | CO₂ Concentration (ppm) |
|---|---|---|
| 1 | 5 | 713 |
| 2 | 10 | 878 |
| 3 | 15 | 930 |
| 4 | 20 | 912 |
| 5 | 25 | 898 |
| 6 | 30 | 874 |
| 7 | 35 | 875 |

Average CO₂ Concentration (ppm) = 895

Accordingly, without Product Packages, the average CO2 concentration naturally produced by 1,000 grams of garlic (i.e. on its own) was found to be 19,265 ppm. With Product Packages absorbing continuously for 72 hours, the average CO2 concentration of each of the 3 consecutive days, for 1,000 grams of garlic, was found to be:
1st day (0 h to 24 h)=2,578 ppm CO2
2nd day (24 h to 48 h)=4,332 ppm CO2
3rd day (48 h to 72 h)=5,141 ppm CO2

Hence, for a 1000-gram batch of garlic over an elapsed duration of 72 hours (i.e. the same bags running continuously over 3 consecutive days), the inventive Product Packages reduced the average CO2 concentration from 19,265 ppm CO2 to 2,578 ppm CO2 in 1st day (0 h to 24 h), 4,332 ppm CO2 in 2nd day (24 h to 48 h), and 5,141 ppm CO2 in 3rd day (48 h to 72 h). Accordingly, the 2 Product Packages with 4 grams of absorber within each absorbed 87% of CO2 gases in 1st day, 78% in 2nd day, and 73% in 3rd day.

### Experiment G: Mangos

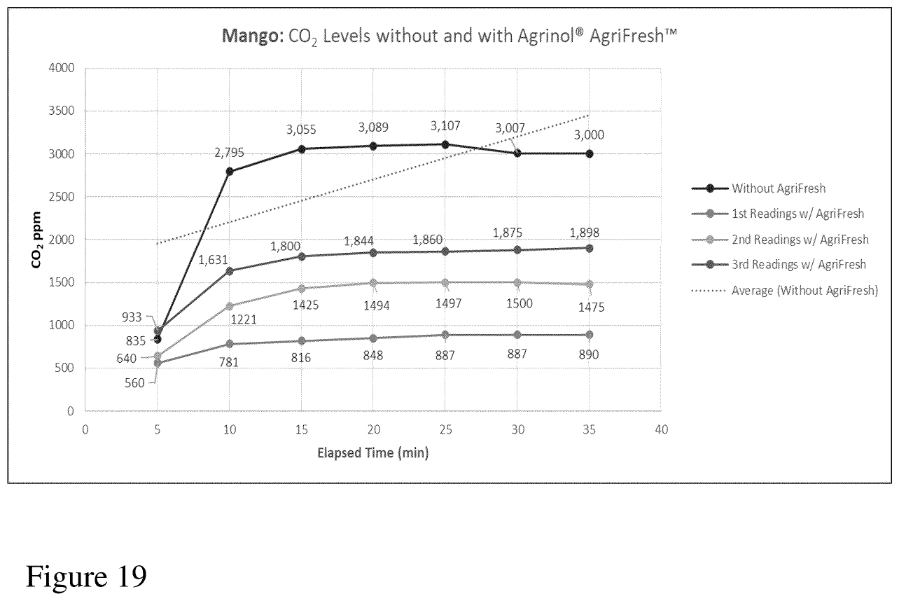

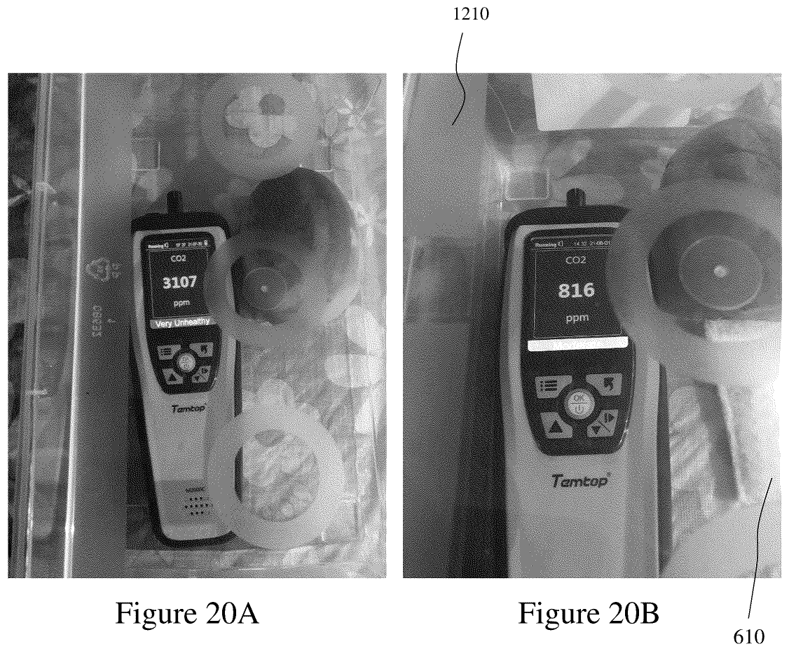

Referring to FIG. 19 there is depicted a graph of the short term evolution of carbon dioxide levels of packaged mangos with and without a Product Package according to an embodiment of the invention. FIGS. 20A and 20B depict packaged mangos with and without a Product Package according to an embodiment of the invention with a sensor device depicting the carbon dioxide levels. A Housing 610 being visible within the Container 1210 within which the Housings 610, the mango and the CO2 Meter were placed.

Produce
Quantity=1
Total Weight=296 grams
Retail Box Size=30 cm×17 cm×11 cm

Table 25 below presents the readings of carbon dioxide without an embodiment of the invention within the Retail Box. 7 measurements were taken, each 5 minutes apart, wherein the first reading (i.e. at 5 minutes elapsed) was ignored to allow for equipment calibration and stability.

TABLE 25
Carbon Dioxide versus Time for Mangos
without Embodiment of the Invention

| Reading No. | Elapsed Time (min) | CO₂ Concentration (ppm) |
|---|---|---|
| 1 | 5 | 835 |
| 2 | 10 | 2,795 |
| 3 | 15 | 3,055 |
| 4 | 20 | 3,089 |
| 5 | 25 | 3,107 |
| 6 | 30 | 3,007 |
| 7 | 35 | 3,000 |

Average CO₂ Concentration (ppm) = 3,009

Accordingly, the average CO2 concentration naturally produced by 296 grams of mangos was 3,009 ppm in the 30 minutes. Accordingly, the short-term average CO2 concentration naturally produced by is 10,165 ppm/kg.

Table 26 below presents the readings of carbon dioxide with 2 Product Packages placed within the vicinity of the produce after the mango and Product Packages had been in the container for 24 hours. Each Product Package containing 4 grams of powder. Again the initial reading was ignored.

TABLE 26
Carbon Dioxide versus Time for Mangos with
Embodiment of the Invention after 24 Hours

| Reading No. | Elapsed Time (min) | CO₂ Concentration (ppm) |
|---|---|---|
| 1 | 5 | 560 |
| 2 | 10 | 781 |
| 3 | 15 | 816 |
| 4 | 20 | 848 |
| 5 | 25 | 887 |
| 6 | 30 | 887 |
| 7 | 35 | 890 |

Average CO₂ Concentration (ppm) = 852

Accordingly, the average CO2 concentration naturally produced by 296 grams of mangos with the pair of Product Packages was 852 ppm in the 30 minutes. Accordingly, the short-term average CO2 concentration in the vicinity of the mango with the Product Packages was 2,877 ppm/kg.

Repeating the measurements after 48 and 72 hours yielded the results in Tables 27 and 28 below. Accordingly, after 48 hours the carbon dioxide concentration from a nominal 1 kg of mangos was reduced to 4,849 ppm and after 72 hours to 6,142 ppm.

TABLE 27
Carbon Dioxide versus Time for Mangos with
Embodiment of the Invention after 48 Hours

| Reading No. | Elapsed Time (min) | CO₂ Concentration (ppm) |
|---|---|---|
| 1 | 5 | 640 |
| 2 | 10 | 1,221 |
| 3 | 15 | 1,425 |
| 4 | 20 | 1,494 |
| 5 | 25 | 1,497 |
| 6 | 30 | 1,500 |
| 7 | 35 | 1,475 |

Average CO₂ Concentration (ppm) = 1,435

TABLE 28
Carbon Dioxide versus Time for Mangos with
Embodiment of the Invention after 72 Hours

| Reading No. | Elapsed Time (min) | CO₂ Concentration (ppm) |
|---|---|---|
| 1 | 5 | 933 |
| 2 | 10 | 1,631 |
| 3 | 15 | 1,800 |
| 4 | 20 | 1,844 |
| 5 | 25 | 1,860 |
| 6 | 30 | 1,875 |
| 7 | 35 | 1,898 |

Average CO₂ Concentration (ppm) = 1,818

Accordingly, without Product Packages, the average CO2 concentration naturally produced by 1,000 grams of mangos (i.e. on its own) was found to be 10,165 ppm. With Product Packages absorbing continuously for 72 hours, the average CO2 concentration of each of the 3 consecutive days, for 1,000 grams of mangos, was found to be:
1st day (0 h to 24 h)=2,877 ppm CO2
2nd day (24 h to 48 h)=4,849 ppm CO2
3rd day (48 h to 72 h)=6,142 ppm CO2

Hence, for a 1000-gram batch of mangos over an elapsed duration of 72 hours (i.e. the same bags running continuously over 3 consecutive days), the inventive Product Packages reduced the average CO2 concentration from 10,165 ppm CO2 to 2,877 ppm CO2 in 1st day (0 h to 24 h), 4,849 ppm CO2 in 2nd day (24 h to 48 h), and 6,142 ppm CO2 in 3rd day (48 h to 72 h). Accordingly, the 2 Product Packages with 4 grams of absorber within each absorbed 72% of CO2 gases in 1st day, 52% in 2nd day, and 40% in 3rd day.

### Summary

From the results presented above for Experiments A through G and as depicted in FIGS. 7 to 20B respectively the CO2 absorption by Product Packages ranged between 40% to 88% across the seven tests, where the rate of natural production and release of carbon dioxide by the produce varied greatly across the different type of fruit and vegetable employed. From these results for the first 5 test cases (apples, red peppers, red potatoes, red onions, and yellow onions) a natural release of carbon dioxide ranging from 1,393 ppm (lower bound) and 3,963 ppm (upper bound) was established which was reduced through the use of the Product Packages in the range of 59% to 88% of emitted carbon dioxide. The last 2 test cases (garlic and mangos) showed a very high natural release of carbon dioxide of 19,265 ppm from garlic, and 10,165 ppm from mango. In these 2 cases, the two small Product Packages employed still absorbed a significant amount of carbon dioxide in the range of 40% to 87% continuously over an elapsed period of 72 hours.

Accordingly, embodiments of the invention, Product Packages, employing a food grade powdered absorber (calcium hydroxide) within permeable membrane bags, absorb substantial amounts of CO2 ppm emissions from all produce within the test scenarios presented.

Accordingly, within the experiments described in respect of FIGS. 7 to 20B the Product Package embodiment of the invention represents one embodiment of the Housing 610, a bag providing a porous membrane within which the absorber material is placed. Within these experiments the powder within the Housing 610 was calcium hydroxide. However, other carbon dioxide absorbers may be employed without departing from the scope of the invention.

Within the following description the term Housing is employed to define a container, portion of a container etc. within which an absorber is placed in order to absorb carbon dioxide and extend the shelf life of produce within storage, retail, domestic, commercial and other parts of the produce food cycle from harvest to consumption. The Housing 610 in FIG. 6 being a specific embodiment of such a Housing. A Housing may within the scope of this specification be singular or it may be plural wherein a Housing may comprise multiple Housings discretely placed with respect to produce or formed into a common assembly for use in association with the produce.

The Housing may comprise a permeable membrane over all surfaces of the Housing or only some surfaces of the Housing.

Within embodiments of the invention the Housing may be placed within a storage container for produce. Within embodiments of the invention the Housing may form part of a storage container for produce wherein the permeable membrane is between the Housing and the inner portion of the container within which the produce is deployed.

Optionally, the Housing may be designed to fit within a slot, or other element, of a storage container such that the permeable membrane is between the Housing and the inner portion of the container within which the produce is deployed. Accordingly, the Housing may be periodically replaced without replacing the container. The container may be a discrete container or a drawer, shelf or other portion of a refrigerator or other storage system for produce.

Within embodiments of the invention the Housing may be placed upon a surface and the produce placed upon the Housing.

The Housing may contain a permeable membrane or multiple permeable membranes only on the surface onto which the produce is placed.

Within embodiments of the invention the Housing may contain an opening, sealable or non-sealable, allowing access to a pocket or pockets within the Housing allowing produce to be placed within the Housing and either sealed or not sealed.

The Housing may contain a permeable membrane or multiple permeable membranes only on the inside of the Housing on the surfaces of the pocket or pockets within which the produce is placed.

The Housing may contain a permeable membrane or multiple permeable membranes which are covered initially by a peelable or otherwise removable cover such that during manufacture, storage and sale of the Housing the powder within the Housing does not absorb carbon dioxide until the peelable or otherwise removable cover is removed.

Within embodiments of the invention the Housing may be thin, e.g. a sheet, such that the produce is wrapped up within the Housing. The Housing may contain a permeable membrane or multiple permeable membranes only on one or more surfaces against which the produce is placed before being rolled or wrapped within.

Optionally, the Housing (also referred to as a housing within the following description) may be contoured and/or shaped to provide recesses or indentations for produce to sit within.

Optionally, a Housing may contain activated carbon in addition to calcium hydroxide or other carbon absorbing powder in order for the Housing to combine an absorber of carbon dioxide and an absorber of odours within confined storage spaces such as containers, refrigerators, shelving, etc.

The used/unused calcium hydroxide, calcium carbonate, and calcium bicarbonate may be disposed of in an environmentally safe manner as it can be used as a fertilizer. Accordingly, depending upon the material(s) employed for the Housing 610 then the used/unused calcium hydroxide, calcium carbonate, and calcium bicarbonate may be disposed of directly or after removal from the Housing 610. For example, if the Housing 610 is formed from biodegradable material(s) then the Housing 610 may be composted, processed etc. for use as a fertilizer or as part of a fertilizer. It would be evident that the Housing 610 with the used/unused calcium hydroxide, calcium carbonate, and calcium bicarbonate or the used/unused calcium hydroxide, calcium carbonate, and calcium bicarbonate discretely can be composted or processed with produce that has become spoilt/rotten. Optionally, a Housing 610 may be disposed within environmental recycling containers, commonly known as “Green Bins” in Canada, to absorb carbon dioxide during the time that produce is within these recycling containers or subsequent transportation, storage, and/or processing stages.

Whilst the embodiments of the invention have been described above with respect to produce it would be evident to one of skill in the art that embodiments of the invention may be employed in conjunction other products, materials etc. where a reduction in carbon dioxide emissions is required during storage, transportation, etc.

The initial experiments performed by the inventors have demonstrated that produce coated with the calcium hydroxide solution according to an embodiment of the invention experienced an increased lifespan compared to their uncoated versions (that are normally found on store shelves). The increased lifespan being a doubling or trebling where most produce without the solution applied lasted for a week before spoiling whilst produce with the solution coating it remained in excellent condition for a month before showing any signs of spoilage. The lifespan and longevity improvements from the application of the solution varies from fruit to fruit and vegetable to vegetable. FIGS. 1B, 2B, 3B and 4B were obtained by spraying the respective produce with a solution of calcium hydroxide.

FIGS. 5A and 6A were obtained by storing the respective produce in proximity to a Housing with a permeable membrane filled with calcium hydroxide powder as were FIGS. 7 to 20B respectively.

As noted with Equations (4A) and (4B) this allows carbon dioxide to be captured either blocking it from reaching the fruit or limiting its release from the fruit. For example, bacteria grow on the surface of crops, absorb CO2 gases, and grow faster depending on the quantity of CO2 exposure. Accordingly, the calcium hydroxide solution absorbs carbon dioxide thereby reducing the carbon dioxide reaching the colonies of bacteria.

Within embodiments of the invention the solution can be sprayed or misted onto produce at various stages of storage, transport, display etc. Within retail environments, the solution may be sprayed/misted periodically onto produce using the misting/spray systems already in place in many such retail environments.

Further, as noted with Equations (5A) and (5B) further carbon dioxide may be absorbed through the reaction of calcium carbonate and water. Accordingly, subsequent misting with water may support this process.

Deployment scenarios for solutions and/or powders according to embodiments of the invention may vary with the vegetable, vegetables, fruit, herb or herbs being stored and fruit to fruit, vegetable to vegetable, and herb to herb.

Deployment scenarios for solutions and/or powders according to embodiments of the invention may vary for different storage and/or transport environments such that deployment configurations for residential storage, e.g. a refrigerator, may be different to that of a retail outlets display area, retail storage area, shipping container etc.

Accordingly, each deployment scenario may include one or more of spraying/misting with a liquid according to an embodiment of the invention, a powder according to an embodiment of the invention, a housing according to an embodiment of the invention, etc.

Within another embodiment of the invention, the inventors have also established the application of a pectin extract to produce to slow down ripening and spoilage of produce.

Optionally, a combination of pectin extract and calcium hydroxide solution may be applied to the produce.

Optionally, the calcium hydroxide solution may be combined with a gelling agent or another component such that the calcium hydroxide solution forms a film over the produce. Accordingly, the produce may be dipped, sprayed with the solution to form a film coating eliminating or reducing the requirement for misting, spraying the produce upon shelves etc. or during storage.

Within embodiments of the invention a mixture of calcium hydroxide and water may be sprayed directly onto fruits, vegetables and herbs, whether manually or automatically in the water misters already installed in grocery stores.

Within embodiments of the invention, a mixture of calcium hydroxide and water may be sprayed on the bed where fruits, vegetables and herbs lie, such as store shelves.

Within embodiments of the invention, a housing comprising an air or air and liquid permeable membrane containing calcium hydroxide and/or calcium carbonate powder may be placed upon store shelves alongside fruits, vegetables, and herbs. Such a housing may form a thin sheet upon which the fruit and/or vegetables and/or herbs are placed. Such a housing may form a storage and/or transport container for the fruit and/or vegetables and/or herbs.

Optionally, the housing may be placed within a storage container of the fruit and/or vegetables and/or herbs. For example, the housing may be placed within a lid of the storage container which has allows air flow from the internal volume of the storage container to the housing. Optionally, the housing may be placed in the storage container with the fruit and/or vegetables and/or herbs. Optionally, the housing may be placed beneath the fruit and/or vegetables and/or herb within the storage container.

Optionally, the housing may form a predetermined portion of a storage container of the fruit and/or vegetables and/or herbs. For example, the housing may be a replaceable portion of the storage container such as its lid for example.

Whilst a single housing has been described it would be evident that multiple housings may be employed.

It would also be evident that the volume of the powder employed within a housing may be varied such that different housings may be employed for different volumes and/or weighs and/or types of produce. Optionally, a large housing with multiple permeable membranes and a larger volume of powder may be employed to provide carbon dioxide capture over an extended period of time before the housing is replaced. Alternatively, small housings may be used at a higher rate of consumption.

Optionally, the housing may be part of product packaging a consumer purchases produce in.

Optionally, the housing may be part of a storage container, storage bag etc. used for temporarily storing produce in.

Optionally, the housing may be part of a drawer, cupboard, shelf, etc. within a domestic, retail or commercial environment such as for storage or transportation.

Optionally, the housing may be part of a drawer, cupboard, shelf, etc. within a refrigerator or food storage system.

Within the experiments presented above in respect of FIGS. 7 to 20B, two or three Agri Fresh™ bags (Housings of which Housing 610 is one embodiment of the invention) were employed each containing only 4 grams of absorber. It would be evident that within embodiments of the invention the amount of powder employed/deployed may be fixed (e.g. part of a container, shelf, etc.) or may be adjusted/established in dependence upon the volume/weight/type of produce.

Accordingly, within embodiments of the invention a first Housing with a first volume of powder may be sold for use with a first predetermined range of produce with volume/weight range(s). Similarly, a second Housing with a second volume of powder may be sold for use with either a second predetermined range of produce with volume/weight range(s) or the first predetermined range of produce with different volume/weight range(s) to that of the first Housing.

Within embodiments of the invention a Housing may contain an electronic circuit in conjunction with an indicator (e.g. multi-colour light emitting diode (LED)) or indicators (e.g. LEDs) and a carbon dioxide sensor wherein the electronic circuit monitors the carbon dioxide level and provides an indication of the Housing's “health” or “status” via the indicator(s). Accordingly, when initially placed with produce, for example, into a crisper drawer or on a shelf of the refrigerator the electronic circuit tracks the evolution of carbon dioxide as the powder absorbs it. However, should the produce remain within the Housing for too long or the Housing has been used/reused multiple times then the efficacy of the powder within the Housing may be reduced thereby triggering an indication to a user that the Housing has reached a point at which it should be replaced and/or the produce removed. Optionally, the electronic circuit may contain a transmitter allowing a communication to be pushed from the Housing to an electronic device, e.g. a refrigeration system, a user's electronic device, remote monitoring system etc. such that any indication is pushed to a local system, remote user or remote system.

Optionally, within other embodiments of the invention the electronic circuit, sensor, and at least one of an indicator and a communications interface may form part of a system associated with the storage and/or transportation of produce or another product for which carbon dioxide reduction is required. This may, for example, be part of a refrigeration system or storage system, a removable portion of a refrigeration system or storage system such as a drawer, shelf, etc.

Optionally, within other embodiments of the invention the electronic circuit and at least one of an indicator and one or more communications interfaces may form part of a system associated with the storage and/or transportation of produce or another product for which carbon dioxide reduction is required. The electronic circuit may via a first communications interface obtain data from a sensor forming part of a housing such that multiple housings can communicate to the electronic circuit and multiple packages of produce or multiple locations of produce monitored by a single electronic circuit which may provide visual indications via the indicator or push notifications via the communications interface or another communications interface. For example, the communication interfaces may be wireless.

Optionally, a Housing may be deployed within an environment in conjunction with separate environmental monitoring of carbon dioxide levels in the vicinity of the produce in order to provide data to a consumer, retailer, shipper, producer etc. as to the current effectiveness of the Housing. Optionally, a software application (e.g. a discrete application or cloud based application) in execution upon a PED, FED or wearable device may obtain sensor data through a wireless interface operating according to a wireless standard and/or a wired interface operating according to a wired standard. The software application in dependence upon a profile relating to the produce to which the sensor(s) are associated may then determine whether the Housing is achieving the desired level of reduction in the carbon dioxide (or other chemical being absorbed) based upon the produce the Housing is associated with, the volume/weight/quantity of the produce etc., the ambient environment, etc. The software application may trigger an alarm, indicator etc. to reflect the status, performance, etc. or it may indicate a requirement to change the Housing, a projected lifetime of the Housing etc. Optionally, a Housing may include a sensor and data log the ambient environment in order to provide a record of the environment of the produce associated with the Housing over a portion of its life cycle.

The exploitation of carbon hydroxide powder and activated carbon powder can also, within other embodiments of the invention, be applied to at least one of storing and shipping at least one of a plurality of seeds and a plurality of seedlings with a Housing. The Housing (container) as in other instances of the invention comprises a casing, a permeable membrane forming a predetermined portion of the casing, the calcium hydroxide powder, and/or and activated carbon powder placed within the container. Accordingly, the calcium hydroxide and activated carbon powder absorbs at least one of moisture, carbon dioxide, and odours within the vicinity of the plurality of seeds and the plurality of seedlings. Further, the calcium hydroxide will kill harmful insects and fungal diseases that can destroy seeds during storage.

The reduction in at least one of moisture and carbon dioxide within the vicinity of the plurality of seeds and the plurality of seedlings reduces at least one of a rate of germination of the plurality of seeds and a rate of root spoilage of the plurality of seedling.

### 2. Coffee Waste Recycling

Coffee is a brewed drink prepared from roasted coffee beans, the seeds of berries from certain Coffea species. When coffee berries turn from green to bright red in color, indicating ripeness, they are picked, processed, and dried. The dried coffee seeds (referred to as “beans”) are roasted to varying degrees, depending on the desired flavor, and then brewed with near-boiling water to produce the beverage known as coffee. The resulting byproduct is commonly referred to as coffee grounds which is acidic.

Whilst coffee grounds contain several key minerals for plant growth, such as nitrogen, calcium, potassium, iron, phosphorus, magnesium and chromium and may also help absorb heavy metals that can contaminate soil. Coffee grounds are acidic. This being evident from Table 29 below where coffee grounds added to water reduce the pH in proportion to the amount of coffee grounds added. Table 29 show that every 10 grams of coffee grounds added to 1 litre of water reduces the pH by 0.25 as the coffee grounds are increased to 50 grams.

TABLE 29
Acidity Variation of Water - Coffee Ground Solution

| Test | Solution | pH | pH Change |
|---|---|---|---|
| 1 | 1 litre (L) H₂O | 7.75 | — |
| 2 | 1 L H₂O + 10 g coffee grounds | 7.52 | −0.23 |
| 3 | 1 L H₂O + 20 g coffee grounds | 7.20 | −0.32 |
| 4 | 1 L H₂O + 30 g coffee grounds | 6.95 | −0.25 |
| 5 | 1 L H₂O + 40 g coffee grounds | 6.71 | −0.24 |
| 6 | 1 L H₂O + 50 g coffee grounds | 6.50 | −0.21 |

| | |
|---|---|
| Average pH Change per 10 g coffee grounds | −0.25 |

Embodiments of the invention have been established by the inventors by way of a liquid formulation and a solid formulation employable in pellet or tablet form.

Liquid Formulation: Within embodiments of the invention, coffee grounds are mixed with a solution of calcium hydroxide and then added to manure. Accordingly, the liquid solution directly absorbs carbon dioxide from the manure, resulting in a powerful mulch that doubles as a fertilizer for all soil types. By providing vital nutrients and balancing the soil's pH, the liquid solution can yield larger crop yields as well as reducing the carbon dioxide released from manure.

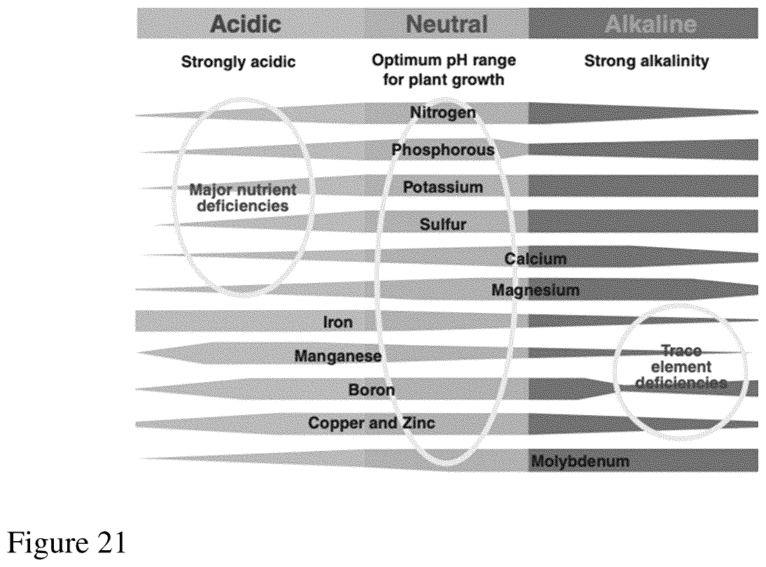

Referring to FIG. 21 there are depicted, schematically, the effectiveness of nutrient uptake by plants from acidic soils through neutral to alkaline soils. Accordingly, it is evident that for most plants, a neutral soil is optimal whilst high acidity leads to major nutrient deficiencies, nitrogen, phosphorous, potassium, sulphur, calcium and magnesium, whilst highly alkaline soils typically result in trace element deficiencies.

Most soils across Canada are acidic (pH between 4 and 5), and coffee waste is also acidic (pH of around 6), simply applying coffee waste as a fertilizer does nothing to address the soil's pH. However, the inventors solution is alkaline (pH between 10 and 11), which balances the soil pH to between 7 and 8. By balancing the pH, vital nutrients (such as nitrogen, phosphorus, and potassium), within plants become more accessible for plant uptake, resulting in larger crop yields. This is because most elements are not ionizable between a pH of 4 to 6, so nutrients are less accessible by plants, hindering peak efficiency and growth.

Accordingly, through the addition of calcium hydroxide (Ca(OH)2) without liquid manure, dry manure, or compost, the pH of a mixture (water+Ca(OH)2+urea+coffee) can be further adjusted by adding a specific amount of an acid, e.g. citric acid, to adjust the final solution pH to between 7.5 and 8.5, allowing it to be used discretely.

The typical solution pH for the mixture (calcium hydroxide+urea+coffee+water) is between 12 and 12.5 which is too high for as outlined above and accordingly the pH solution should be adjusted to between 7 and 8. Accordingly, the addition of an acid or acids reduces the pH to between 7 and 8, which is in the right range for the soil to not impact the uptake of nutrients by plants growing in the soil.

Optionally, within embodiments of the invention citric acid may be employed as the acid.

Optionally, within embodiments of the invention humic acid may be employed as the acid.

Optionally, within embodiments of the invention citric acid and humic acid may be employed in combination as the acids.

Humic acid(s), in addition to providing pH adjustment, is an excellent additive for soil and plants through its ability to increase ion exchange (cation exchange) between the soil and plant roots. Humic acid(s) also help to increase soil microorganisms' activities for plants to grow better and helps soils to retain moisture and help reduce nutrient loss. Humic acid(s) are typically diluted with water, a typical ratio being 1:10 (humic acid/water). Humic acid can be applied at a rate of approximately 50 ml (approximately 1.7 fluid ounces).

Optionally, humic acid(s) may be used discretely. Optionally, humic acid(s) may be used in conjunction with one or more other acids including, for example, citric acid.

Accordingly, the addition of the solution according to embodiments of the invention provides for absorption of CO2 from the manure as well as balancing the pH of the soil to which it is applied. Accordingly, the inventor's solution allows for improved access to nutrients.

Coffee bean suppliers who use soil to grow their beans can easily implement this system into their daily routines. Utilizing coffee waste from coffee vendors, which would otherwise accumulate in landfills, as fertilizer to grow more coffee beans closes a loop of product life by creating a complete and efficient cycle of life. This eliminates the dumping of coffee waste into landfills. Alternatively, rather than transporting the coffee waste back to the locations of coffee production, the coffee waste may be employed with the manure locally to the coffee waste production to improve production of plants, vegetables and/or fruit. Beneficially, embodiments of the invention whilst enhancing agriculture also allow coffee vendors to demonstrate use of an environmentally-conscious, locally-sourced, green technology who actively seek sustainable methods, strengthening our economy.

The following process represents an exemplary sequence for generating and employing an additive to organic waste material:
Calcium Hydroxide Ca(OH)2, for example, 1.5 to 2 grams per liter of water;
Coffee Waste, for example, 1 kilogram of coffee grounds per 4 liters of final solution.

An optional additive to the coffee ground-calcium hydroxide mixture may include urea (source of nitrogen), for example, added at 1 gram per liter of mixture.

An optional additive to the coffee ground-calcium hydroxide mixture may include an antifungal agent.

An optional additive to the coffee ground-calcium hydroxide mixture may include gum acacia, or another material, for use as a binder. Gum Arabic (gum acacia) is a complex mixture of glycoproteins and polysaccharides predominantly consisting of arabinose and galactose such that it acts as a glue or binder.

An optional additive to the coffee ground-calcium hydroxide mixture may include a gelling/thickening agent. One such agent being gelatin for example.

Within exemplary embodiments of the invention, the inventors have produced organic fertilizers which differ in the ratios of their constituents.

### Mixture A: Dry Manure

Mixture Ratio: Coffee (kilogram):Solution (liters):Manure (kilogram)
Mixture Ratio: 1:4:4
Mixture pH: 8:0
e.g. 1 kg of coffee grounds, 4 liters of solution; and 4 kg of manure.

### Mixture B: Compost

Mixture Ratio: 1:5:4
Mixture pH: 7.2
e.g. 1 kg of coffee grounds, 5 liters of solution; and 4 kg of manure.

### Mixture C: Liquid Manure

Mixture Ratio: 1:2:4
Mixture pH: 7.85
e.g. 1 kg of coffee grounds, 2 liters of solution, and 4 liters of liquid manure

Manufacturing requires that the solution be mixed well, for dissolvability and solubility, and to avoid exposure to air during mixing as much as possible (to prevent undesired early reaction). It is important for the containers to be sealed once filled to also prevent undesired early reaction.

It would be evident that as the organic matter to which the mixture is added may vary from farm to farm, the exact ratio may differ depending on the pH of the manure or compost, but the overall recipe remains the same.

Solid Formulation: Alternative to a liquid formulation the inventors have established a solid formulation for use as pellets or tablets, for example, to recycle coffee waste with a fertilizer. Accordingly, the solid formulation comprises a series of main components together with one or more binding agents. The main components of the solid formulation being coffee grounds, calcium hydroxide powder, and manure and/or urea. The one or more binding agents may be selected from, but not be limited to, the group comprising cornstarch, acacia, tragacanth, gelatin, starch paste, pregelatinized starch, alginic acid, cellulose, sucrose, and liquid glucose.

Initial experiments by the inventors have established a series of formulations, of which four exemplary formulations are outlined below in Table 30.

Four examples of combinations and ratios we have tested so far are:

TABLE 30
Exemplary Solid Formulations

| | | | Calcium | | |
|---|---|---|---|---|---|
| | | Coffee | Hydroxide | Binder | |
| | Manure | Grounds | (Ca(OH)₂) | (Cornstarch) | Water |
| Formulation | (g) | (g) | (g) | (g) | (mL) |
| 1 | 50 | 50 | 0.50 | 0.50 | 0 |
| 2 | 50 | 50 | 0.50 | 0.50 | 330 |
| 3 | 50 | 50 | 0 | 0.50 | 0 |
| 4 | 0 | 10 | 0 | 0.50 | 0 |

It would be evident to one of skill in the art that the exemplary formulations above represent a subset of the formulations possible within the scope of the invention and that these and others may be appropriately scaled according to the size of the pellet/tablet or other form of the solid product for sale, marketing, use, disposal etc.

Among the advantages of employing tablets/pellets or other solid formulations rather than liquid form for coffee recycling are ease of use, ease of transportation, preventing fermentation that causes unpleasant odours and undesired mold (for example), enabling slow release of minerals long-term, and tablets/pellets are pre-measured for the user, this means that the quantity is already adjusted, thus decreasing or eliminating user error. Further, via the provisioning of reference tables with respect to the current pH of a soil within which plants are growing the user can establish the appropriate adjustment of pH to between pH 7 to pH 8 to allow for the most nutrients, elements, and minerals to be soluble as indicated in FIG. 21. This maximizes availability to be absorbed by plant roots, so in this case, the benefits to farmers and environment is to use less chemical fertilizer, allowing plants and trees to grow and have greater yields since they require it.

Within embodiments of the invention the pellets/tablets may further include citric acid or other acidic powder to the mixture containing the coffee.

Within other embodiments of the invention the pellets/tablets may comprise only coffee grounds (coffee waste) without any manure, soil etc. in liquid or solid form, which is then mixed with water, for example. According to an embodiment of the invention the inventors mixed 10 grams coffee with 0.10 grams of calcium hydroxide powder (Ca(OH)₂) and 5 mL of water. The measured pH was initially 9.8 and reduced to 7.8 the next day but when exposed to carbon dioxide the pH reduced further to 7.0. This occurs when the calcium hydroxide absorbs CO2, converting into calcium carbonate & bi-carbonate, which adjusts soil pH, becoming near neutral (pH 7.0).

The inventors have also established a variation of the invention where coffee grounds are replaced with tea waste, e.g. used tea leaves. For example, 4 grams of tea may be mixed with 0.10 grams of calcium hydroxide powder (Ca(OH)₂), 5 mL of water and some cornstarch dough (for example 0.50 grams). The measured pH was initially 9.8 and reduced to 7.8 the next day but when exposed to carbon dioxide the pH reduced further to 7.0.

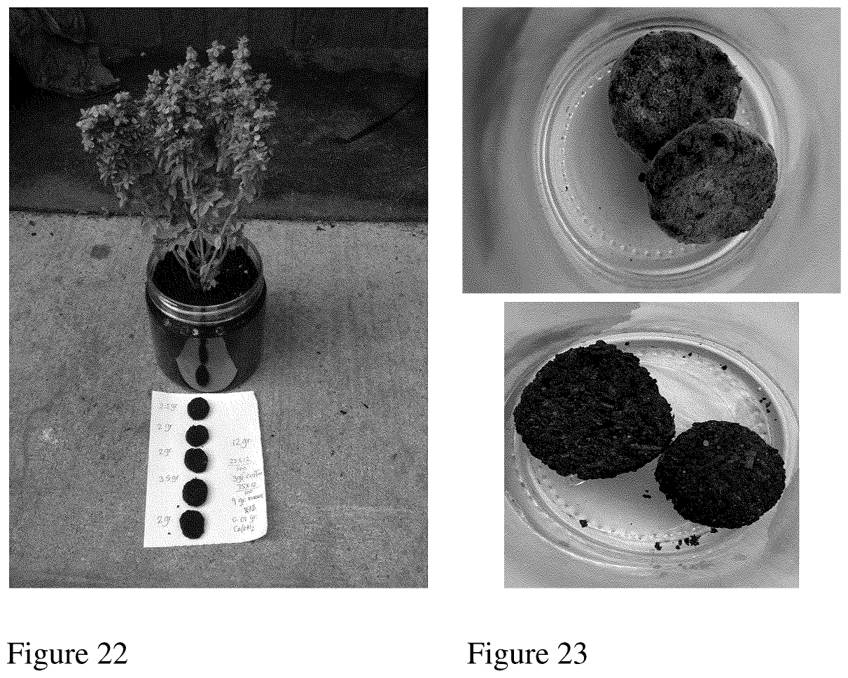

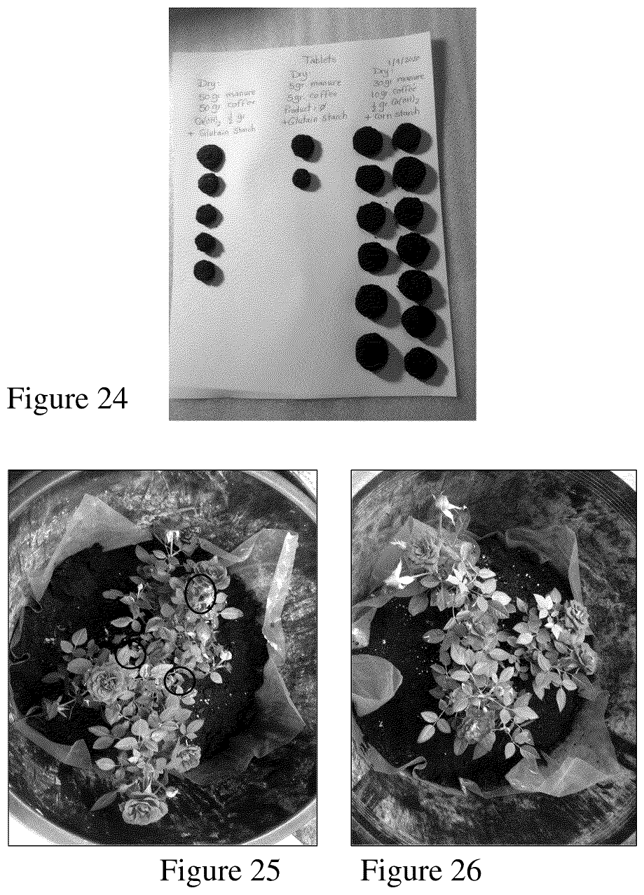

Referring to FIG. 22 there is depicted a basil plant with 5 pellets manufactured by the inventors to provide a total of 12 grams of mixture comprising 3 grams of coffee, 9 grams manure and 0.01 grams of calcium hydroxide powder (Ca(OH)₂). The five tablets having discrete weights as indicated in FIG. 22. FIGS. 23 to 24 depict photographs of pellets formed according to embodiments of the invention wherein FIG. 23 depicts images of pellets, FIG. 24 depicts pellets with three compositions according to embodiments of the invention representing exemplary compositions:
Left (5 pellets): 50 grams manure, 50 grams coffee, 0.50 gram Ca(OH)₂ and gluten starch (0.50 grams);
Middle (2 pellets): 5 grams manure, 5 grams coffee waste and gluten starch; and
Right (13 pellets): 30 grams manure, 10 grams coffee waste, 0.50 gram Ca(OH)₂ and corn starch (0.50 grams).

FIGS. 25 and 26 depict photographs of a rose plant prior to and after application of coffee-based pellets according to an embodiment of the invention using a composition as described above and depicted in FIG. 24. In FIG. 25, discoloration (yellowing) is evident on some leaves which is not evident in FIG. 26 where the topsoil has been augmented with coffee based pellets according to an embodiment of the invention.

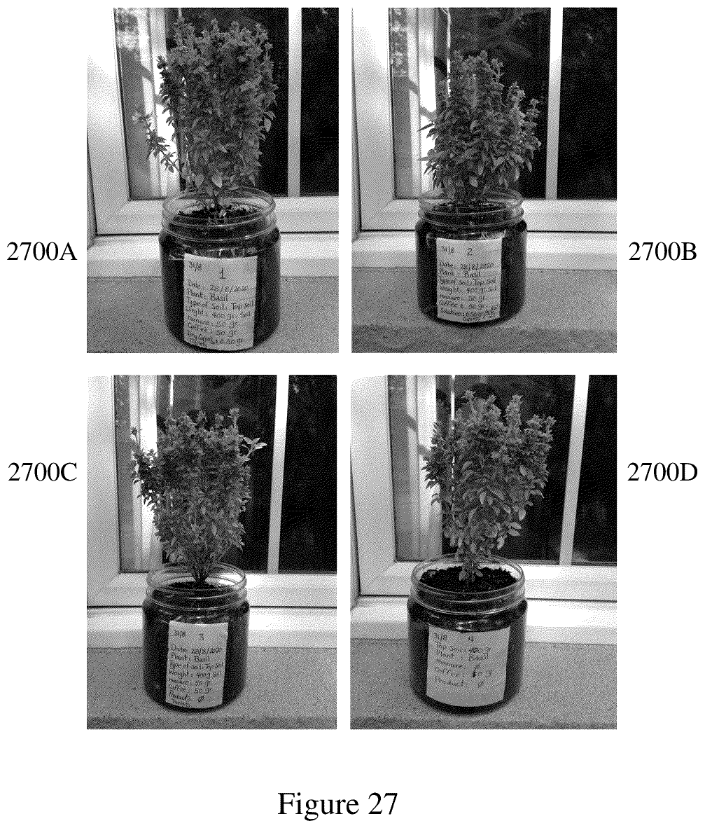

FIG. 27 depicts photographs of basil plants with different pellet combinations added according to embodiments of the invention to provide for adjustment of the pH from the initial level of each plants topsoil towards the target neutral pH of 7. First Image 2700A depicts a first plant where 400 grams of topsoil were mixed with pellets providing 50 grams manure, 50 grams coffee waste, and 0.50 gram Ca(OH)₂ powder (dry). Second Image 2700B depicts a second plant where 400 grams of topsoil were mixed with pellets providing 50 grams manure, 50 grams coffee waste, 0.50 gram Ca(OH)₂, and 330 mL water. Third Image 2700C depicts a third plant where 400 grams of topsoil were mixed with pellets providing 50 grams manure and 50 grams coffee waste. Fourth Image 2700D depicts a fourth plant where 400 grams of topsoil were mixed with 10 grams of coffee waste.

### 3. Destruction of Coronavirus, Bacteria, and Fungus from Air, Floors, and Carpets

Coronavirus disease 2019 (commonly referred to as COVID-19 or simply COVID) is a contagious disease caused by severe acute respiratory syndrome coronavirus 2 (SARS-CoV-2). The SARS-CoV-2 virus cells comprising a nucleic acid inside surrounded by a membrane of fat and protein.

As herd immunity is established, either through infection or immunization, then within the subsequent period as factories and retailers begin to reopen fully, the global carbon dioxide emissions will return to previous levels, or, worse, rise to new heights. It has been reported that air pollution exacerbates the effect of the coronavirus, as it weakens the immune system of the individual, thereby significantly compromising their ability to fight the infection. Even small increases in fine atmospheric particulate matter can have had a catastrophic impact. An increase of just 1 microgram of pollution per cubic metre has been reported to result in a 15% increase in COVID-19 deaths. Globally, 55% of the world's population lives in urban areas, a proportion that is expected to increase to 68% by 2050. Today the most urbanized regions include Northern America (with 82% of its population living in urban areas in 2018), Latin America and the Caribbean (81%), Europe (74%) and Oceania (68%). Within such urban environments, not only does the infection spread faster (or requires dramatic actions such as lockdowns of the population to limit or slow its spread) but these areas have increased atmospheric pollution. Accordingly, the mortality rates as well as overall infections rates are significantly higher in urban areas than in rural areas.

It has been demonstrated that alkaline solutions destroy coronavirus, as a result of their high pH. Common “coronavirus killers,” such as isopropyl alcohol and soap, have pH of around 8 and 9-10, respectively. Since coronavirus does not survive in an alkaline environment of 8 and above, the solutions of the inventors function as a coronavirus killer, effectively destroying coronavirus particles from the air and surfaces. This occurs due to the structure of the virus, which is composed of nucleic acid on the inside and an outer membrane of fat. The virus relies on its membrane of fat for infecting cells. The inventor's calcium hydroxide liquid solution, with a pH of 10 to 11, destroys the outer fatty membrane of coronavirus particles, the same way these other known coronavirus killers do.

Beyond virus destruction, the employment of solutions according to embodiments of the invention can also lead to improved cognitive function. Whilst it has been demonstrated that air pollution particles can serve as vehicles for viral transmission, resulting in disease spread and reduction in cognitive function, research has also shown that students' complex strategic thinking abilities may decrease by 50% by the end of the century as a result of carbon emissions if they continue to increase as projected. The United Nations has suggested that humans must consume less meat and dairy in order to lessen greenhouse gas levels arising from farming but this is not a realistic or sustainable long-term solution when entire industries and their resulting infrastructure, employment, etc. are considered. Further, replacing meat protein generally requires significantly larger areas of agriculture and increased water consumption which are natural resources equally in limited supply.

Accordingly, it would be beneficial to reduce both particulates and carbon dioxide within the residential, commercial, and other environments representing our urban environment. The inventors have demonstrated that their calcium hydroxide solution can reduce by 52% fine inhalable particles, with diameters that are generally 2.5 micrometers and smaller (PM2.5 particulates) and 53% of inhalable particles, with diameters that are generally 10 micrometers and smaller (PM10 particulates) suspended in the air, which are known to support carrying of viral infections, impede breathing, and cause respiratory crises.

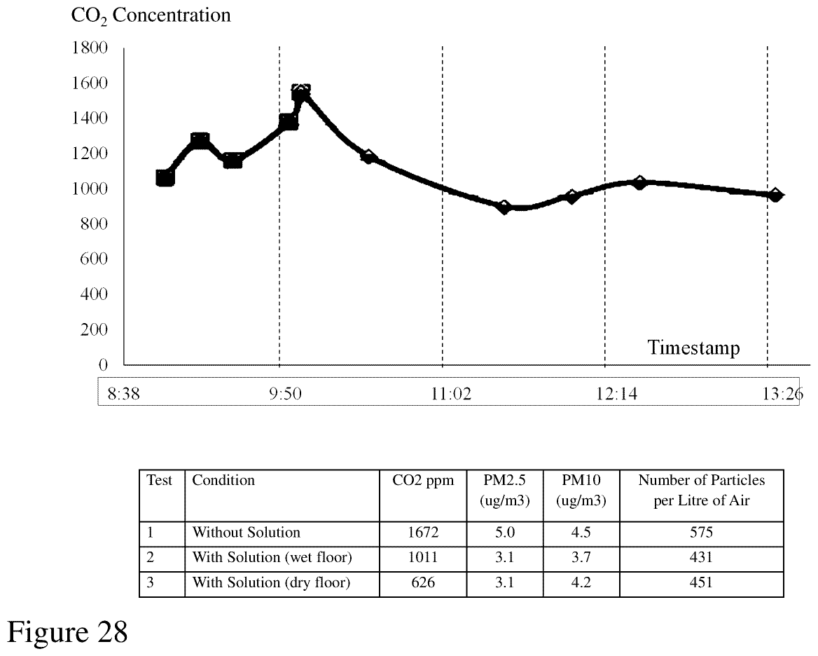

Within an initial experiment, the results of which are depicted in FIG. 28, carbon dioxide levels were measured before product application, from 9 am to 10 am, in 15-minute intervals. CO2 levels showed that CO2 kept rising from 9 am to 10 am, beginning at a concentration of approximately 1100 ppm at 9am and ending at approximately 1590 ppm at 10 am. The inventive product was applied to the floor, mopped, and left to dry naturally (no forced airflow or heat were employed) for 3.5 hours, during which CO2 levels were measured in 30 minute intervals. After product application, carbon dioxide levels dropped from approximately 1590 ppm (measured at 10:00 am) to approximately 990 ppm (measured at 1:30 pm), showing a 38% reduction in carbon dioxide levels over a period of 3.5 hours. The table in FIG. 28 presents the data obtained by the inventors in tabular form.

In order to demonstrate the efficacy of inventive embodiments of the invention the inventors employed an external laboratory to perform measurements upon samples for the enumeration of Escherichia coli and total coliform by membrane filtration. This method allows for the simultaneous detection and enumeration of Escherichia coli (E. coli), total coliforms and background organisms with a single filtration, on a single agar plate (DC media), incubated at one temperature. The method comprising passing a known volume of a sample (e.g., 100 mL) through a 0.45 μm membrane filter using vacuum filtration and applied to a nutrient selective medium. These tests are incubated for 24±2 hours at 35.0±0.5° C. During this incubation period E. coli and total coliform Colony Forming Units (CFUs) will emerge as light blue or dark blue/purple colonies (EC) and red and/or pink colonies (non-E. coli coliforms). Total coliform colonies are the sum of E. coli and non-E. coli coliform counts. All colonies meeting the above criteria are counted and used to calculate the number of Colony Forming Units per 100 mL of sample. Colonies may arise from pairs, chains, clusters or single-cells, all of which are included in the term colony forming units. All remaining colonies (ones that do not fit the E. coli and coliform criteria) are counted as background colonies.

Accordingly, a solution established by the inventors was provided to the external laboratory and subjected to the above measurement process resulting in the results presented in Table 31 below for the sample, reference B20-37485-1.

TABLE 31
Measurement of Sample from External Laboratory

| Parameter | Total Coliform | E. coli |
|---|---|---|
| Units | cfu/100 mL | cfu/100 mL |
| R.L. | 1 | 1 |
| Reference Method | MOE E3407 | MOE E3407 |
| Result | 0 | 0 |

It would be evident that additionally many materials employed within indoor environments, such as carpets, curtains, etc. will collect various colonies of bacteria. Accordingly, such surfaces can be treated through the application of a spray/mist of the inventive solution. The spray/mist may also be applied as a liquid during a wet vacuuming process. Embodiments of the invention may be applied to hard floors, e.g. tiles or wood flooring, soft floorings, e.g. rugs or carpets. Embodiments of the invention may be implemented with handheld machines and floor based machines in residential, domestic, industrial, commercial environments.

Beneficially, with carpets, curtains, etc. the solution will reduce bacterial infections and reduce carbon dioxide. As calcium hydroxide Ca(OH)2 has a pH of 10 to 11 (alkaline), it effectively kills coronavirus cells as well as captures carbon dioxide. This can be implemented in hospitals, schools, offices, factories, airplanes, ships, etc., to effectively destroy coronavirus cells and other viruses/bacteria etc. as well as capturing carbon dioxide from the air. The resulting calcium carbonate can be removed periodically through application of techniques such as vacuuming, beating, washing etc. whilst the water will typically evaporate.

### 4. Acetic Acid Production in the Farming Sector by Utilizing Methane & Carbon Dioxide Gases

Amongst the various environmental issues, climate change, commonly referred to as global warming, is one that has been directly attributed to the so-called greenhouse gases (GHGs), being carbon dioxide (CO2) and methane (CH4), both of which have been increasing as a direct result of human activity at all levels. The farming industry contributes approximately 15% to the global GHG levels due to methane release by animals directly and from decomposition of manure. Accordingly, providing a solution to the CO2 and CH4 problem at the farming level is beneficial but the approaches outlined are implementable anywhere these gases are produced.

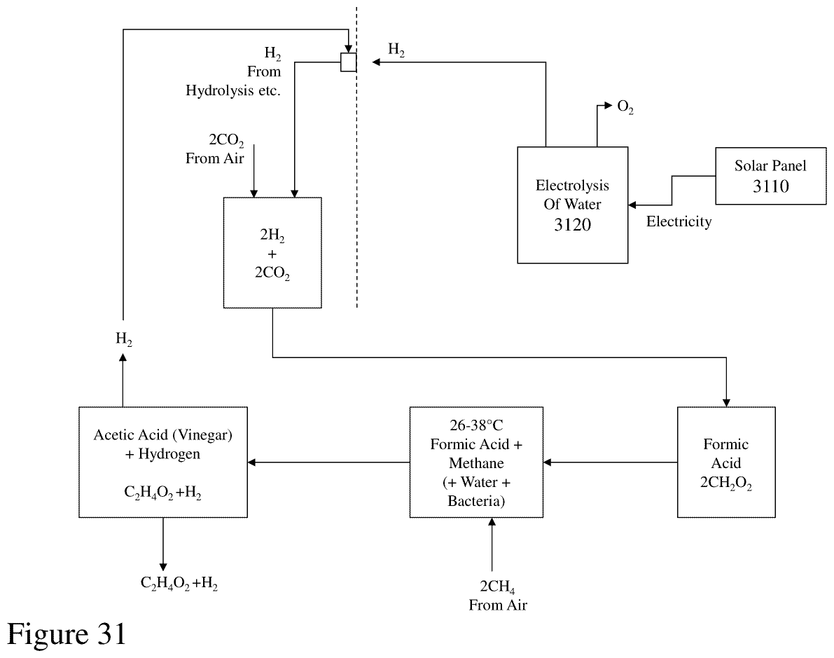

According to an embodiment of the invention, such as depicted by the exemplary schematic in FIG. 31, the method comprises:
Step 1: Mix two molecules of Carbon Dioxide (2 CO2) with two molecules of Hydrogen (2 H2) to generate formic acid as shown in Equations (6A) and (6B);

Carbon Dioxide+Hydrogen→Formic Acid (6A)

2CO₂+2H₂→2CH₂O₂ (6B)
Step 2: Mix Formic Acid CH₂O₂ with methane gas (CH₄) and water (H₂O), at a temperature between 26° C. and 38° C., in the presence of an added bacteria (acetobacter or acetozym) as an aid (or reaction's catalyst), as shown in Equations (7A) and (7B);

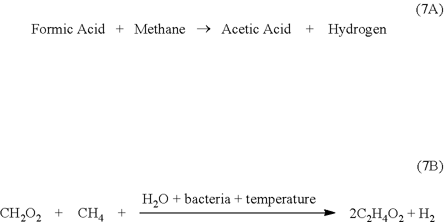

Accordingly, the process results in acetic acid (C₂H₄O₂) (vinegar) and hydrogen gas, which can be re-used. Optionally, the process reactor vessel may be within a manure storage container as manure, as it breaks down, generates heat so that the heat can be directly utilized by the process reactor vessel to eliminate or reduce the requirements for providing additional heat.

Accordingly, methane captured from manure storage etc. can be used to produce vinegar directly at a farm, e.g. dairy or meat producing farm.

Initial production of hydrogen for the reaction given in Equation (3) and providing makeup for losses arising through the processing described above can be implemented through multiple processes. However, two processes that have low environmental impact are electrolysis (which can be driven through solar generated electricity for example) and sugar fermentation. With respect to the electrolysis of water this can be achieved at temperatures between 50° C. and 80° C. This proceeds according to Equation (8) below. An exemplary system for providing electrolysis of water is the Hofmann voltameter although other systems may be employed including, for example, catalytic hydrolysis through semiconductor nanowire devices with catalytic nanoparticle such as those of Mi et al. “High Efficiency Broadband Semiconductor Nanowire Devices” in U.S. Pat. No. 9,112,085, for example. Electrolysis may employ electricity generated from a solar panel, solar cell, wind turbine etc. so that the process can be run with a low carbon footprint. However, it would be evident that alternatively, hydrogen could be sourced in pressurised liquid form and electricity may be procured from an electrical supplier via an electrical grid.

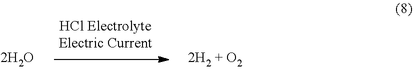

Accordingly, hydrogen gases can be used for acetic acid production as outlined above. Further, the acetic acid has a direct market application in addition to the use of the hydrogen.

### 5. Methane Gas Removal and Capture

As noted above, animal agriculture represents a significant contributor to the emissions of GHGs. The percentage output of methane from major global contributors is:
Fossil fuels 33%
Animal agriculture 30%
Human waste flows 18%
Plant agriculture 15%
All other 4%

Further, methane as a GHG has an impact on global warming potential 30 times that of carbon dioxide over a century timeframe. However, the oxidation of methane as evident from Equations (9A) and (9B) whilst removing the methane generates additional carbon dioxide. However, this carbon dioxide can be reduced through the use of salts hydroxides or elemental hydroxides such as calcium hydroxide [Ca(OH)2], potassium hydroxide (KOH), sodium hydroxide (NaOH), and magnesium hydroxide (MgOH). Calcium hydroxide being particularly beneficial as it results in calcium carbonate which has applications within the construction industry, paints, glazes, adhesives, sealants, fillers, dietary supplement, sugar beet refining, soil neutralizer, and cleaning products. Equations (10A) and (10B) and (11A) and (11B) presenting the conversion of carbon dioxide to calcium carbonate with calcium hydroxide.

Methane+Oxygen→Carbon Dioxide+Water (9A)

CH₄+O₂→CO₂+H₂O (9B)

Carbon Dioxide+Calcium Hydroxide→Calcium Carbonate+Water (10A)

CO₂+Ca(OH)₂→CaCO₃+H₂O (10B)

Calcium Carbonate+Carbon Dioxide→Calcium Bicarbonate+Water (11A)

CaCO₃+CO₂→Ca(HCO₃)₂+H₂O (11B)

The oxidation of methane may be performed through bio-oxidization with methane monooxygenases such as those found in methanotrophic bacteria, for example.

Optionally, hydrogen peroxide (H2O2) may be employed as an oxidizer of the methane before using calcium hydroxide, to convert methane to carbon dioxide.

### 6. Diesel Exhaust

Diesel exhaust fluid (DEF) is a liquid used to reduce the amount of air pollution created by a diesel engine. Specifically, DEF is an aqueous urea solution made with 32.5% urea and 67.5% deionized water. DEF is consumed in a selective catalytic reduction (SCR) that lowers the concentration of nitrogen oxides (NOx) in the diesel exhaust emissions from a diesel engine. The DEF is injected into the exhaust pipeline which is at elevated temperatures through the elevated temperature exhaust gases from the diesel engine.

Urea+Nitrogen oxide+Oxygen→Nitrogen+Water+Carbon Dioxide (12A)

2(NH₂)₂CO+4NO→4N₂+4H₂O+2CO₂ (12B)

Urea+Nitrogen Dioxide→Nitrogen+Water+Carbon Dioxide (13A)

4(NH₂)₂CO+6NO₂→7N₂+8H₂O+4CO₂ (13B)

Accordingly, it is evident that DEF whilst cleaning the diesel exhaust of nitrogen oxide (NO) and nitrogen dioxide (NO₂) results in increased carbon dioxide emissions.

Accordingly, the inventors have established a variant of DEF wherein a solution of calcium hydroxide is employed. As noted above in Equations (9A) and (9B), the calcium hydroxide would react with carbon dioxide resulting in calcium carbonate. As calcium carbonate has a high melting point then the calcium carbonate would be particulate and could be either exhausted into the environment wherein it would settle or be filtered.

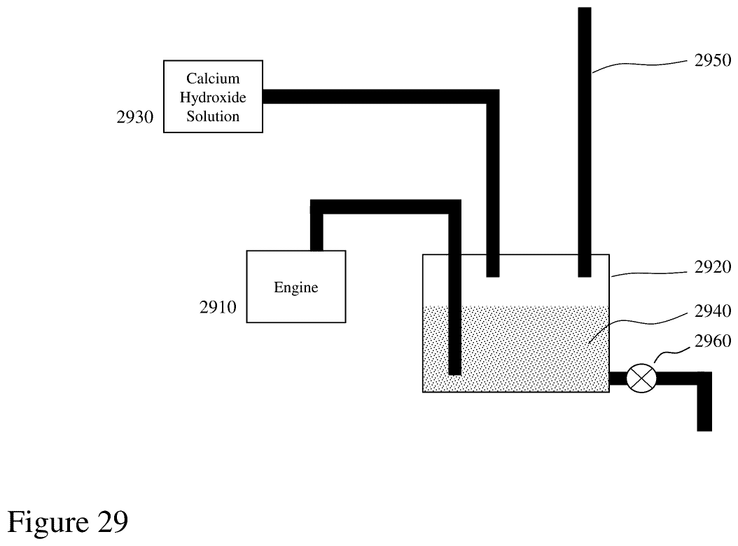

Referring to FIG. 29 there is depicted a schematic of a diesel engine exhaust processing system according to an embodiment of the invention. As depicted an Engine 2910 generates exhaust which is coupled to Processing Tank 2920 which contains a calcium hydroxide solution (Ca(OH)₂) 2940 which is fed to the Processing Tank 2920 from a Storage Tank 2930. The exhaust from the Engine 2910 is bubbled through the Ca(OH)₂ Solution 2940 where the reaction given by Equation (10) proceeds to generate CaCO₃ and Ca(HCO₃)₂ precipitates which can then be flushed from the Processing Tank 2920 via Outlet 2960. The remaining cleaned exhaust gases from the Engine 2910 are vented through Vent 2950.

If the exhaust processing system depicted in FIG. 29 was installed upon a marine craft then the Outlet 2960 may direct the precipitate and water etc. directly into the marine environment. Flushing in this instance may be via extracting and pumping marine water from around the marine craft through the Processing Tank 2920 (this flushing system not being depicted for clarity).

As noted previously above, the resulting byproduct of the alternative DEF according to embodiments of the invention is a material that can be employed/disposed of into the environment safely. In fact, it can be beneficially deployed within agriculture. Further, as the system according to embodiments of the invention is a modest adaptation of the existing exhaust system, it is exhausted via the Processing Tank, the system can be employed discretely in conjunction with existing exhaust systems without modification of the material employed or it can be employed in conjunction with a catalytic converter as it can be subsequent to the catalytic converter. The inventors estimate that 207 kg of carbon dioxide can be removed from the environment per year for an automobile and more for diesel-based trucks etc.

### 7. Animal Feed Additive

Calcium carbonate and calcium hydroxide provide a source of calcium to biological systems, such as animals and humans. This has benefits within agriculture for increasing milk production in dairy cows or beef cattle, etc.

Optionally, these materials may be added as an animal feed pH adjustment thereby helping digest cellulose and food ingredients.

Beneficially, calcium improves hormonal action and could reduce or remove the need to add more hormones to animal feed.

Calcium hydroxide (in powder form) can be used as an animal feed additive can help accelerate and ease animal digestion, for example when mixed with soya bean grains or corn grains and prepared as tablets/pellets, when fed to meat or dairy cattle. These tablets/pellets when mixed with hay, helps increase the animals meat and dairy production, whilst maintaining good animal health.

Additionally, when animals digest the feed constituents then carbon dioxide (CO2) and methane (CH4) are produced via their excrement (manure) and gases. Accordingly, the calcium hydroxide powder placed within the feed acts also as a carbon dioxide absorber thereby absorbing the CO2 generated in the animals digestive processes and turning it into a precipitate expelled with the manure.

Within other embodiments of the invention, methane may also be absorbed if it can be converted to carbon dioxide via an oxidation process. Accordingly, within other embodiments of the invention, the inventors include a further additive, such as potassium permanganate, to function as a catalyst oxidizer for methane in order to facilitate this.

The inventors have also established adding vitamin D (one or more fat-soluble secosteroids) with calcium hydroxide powder to control the levels of calcium and phosphates ions in the blood, as well as calcium and magnesium absorption in the intestines. Magnesium is important for anaerobic respiration.

Further, the calcium hydroxide or calcium carbonate may be combined with coffee grounds into an animal feed in pellet form etc. A beneficial quality of coffee waste is that there is already a reduced amount of caffeine, so animal health and behaviour would not be affected, and this could be further reduced through additional water treatments (e.g. variants of the Swiss water decaffeination process).

### 8: Caulking, Asphalt, Sealants

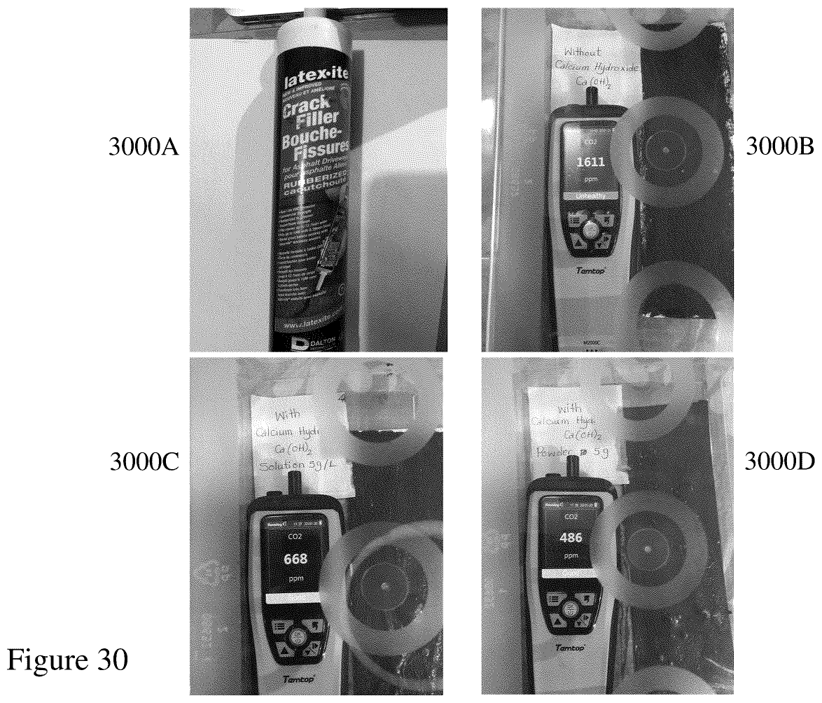

Another use of absorbers according to embodiments of the invention is with respect to the caulking materials such as used and employed in asphalt sealing, driveway sealants, crack fillers, etc. Referring to FIG. 30 there is depicted in first Image 3000A a standard commercial filler tube for dispensing the caulking material, e.g. through a mechanical plunger. Second image 3000B depicts the resulting carbon dioxide levels of the sealant within an enclosed region (in this instance a plastic container) showing a carbon dioxide level of 1611 ppm. Third and fourth images 3000C and 3000D depict the same caulking when placed within the container with a calcium hydroxide solution and calcium hydroxide powder respectively. Third Image 3000C being with a solution comprising 5 grams of calcium hydroxide per litre of water. Fourth Image 3000D being with 5 grams of calcium hydroxide powder.

There are many ways to manufacture asphalt and its components, according to the prevailing weather conditions in each country. However, the inventors have focused on how to reduce what are commonly referred to as “greenhouse gasses” within the asphalt mixture, particularly during its use such as within hot paving processes. Accordingly, carbon dioxide and sulfur dioxide can be reduced by mixing a solution or dry powder according to an embodiment of the invention. Driveway sealant is a petroleum product that can be applied to exterior surfaces including concrete, brick, paving stones, flagstone, aggregate and natural stone for example. These sealants are typically water resistant, UV resistant, and breathable. Physically reactive sealants contain butyl, bitumen, and synthetic rubber, and chemically reactive sealants contain acidic, neutral, and alkaline silicone, which form chemical substances such as acetate, alkoxy, benzamide, enoxy, ester, oxime, and amine. Accordingly, a solution or dry powder according to an embodiment of the invention can be mixed with a driveway sealant to absorb carbon dioxide and sulfur dioxide.

For many applications, an exemplary composition for a liquid solution according to an embodiment of the invention would be to dissolve 3 grams of calcium hydroxide per litre of water. However, in the case of asphalt, a more concentrated solution is employable, with, for example, 5 grams, 7 grams or greater than 7 grams of calcium hydroxide per litre of water as the elevated temperature of asphalt when laid down allows the user of a hotter solution thereby allowing increased calcium hydroxide in the water thereby allowing increased carbon dioxide absorption.

For use with a driveway sealant a powder and/or solution according to an embodiment of the invention (e.g. based upon calcium hydroxide) is employed using for example, 3 grams of calcium hydroxide per litre of water if an increased temperature solution can be employed a more concentrated solution of 5-7 grams or more of calcium hydroxide per litre of water may be employed.

In either instance of asphalt or driveway sealant other ratios may be employed without departing from the scope of the invention. It would be evident that in hotter climates or parts of the year higher temperature solutions can be employed helping to increase the amount of calcium hydroxide and thereby absorb more carbon dioxide and sulfur dioxide.

In the case of using a calcium hydroxide powder for paving, sealing of roofs, etc. the spreading of a dry powder can be employed instead of a solution although a solution could still be employed. In the instance of the powder a solution would be subsequently formed as the powder dissolved within rainwater such that the resulting solution can absorb carbon dioxide and sulfur dioxide from surfaces.

For a caulking agent (e.g. a crack filler) experiments by the inventors yielded the results below. For each reading, a reading was taken at the beginning of and end of the minute. We then took the average of the two readings where the readings were taken after 1 hour.

Scenario 1: Without Calcium Hydroxide:

TABLE 32
Sealant without Calcium Hydroxide

| | | | 1^{st} Reading | 2^{nd} Reading | Average |
|---|---|---|---|---|---|
| Parameter | Value | | (ppm) | (ppm) | (ppm) |
| Sealant | 20 | g | | | |
| Solution | — | | | | |
| Area | 8 × 25 | cm | | | |

| | | | |
|---|---|---|---|
| Reading A | 1,611 | 1,422 | 1,157 |
| Reading B | 1,330 | 1,222 | 1,276 |
| Reading C | 1,200 | 1,114 | 1,157 |
| Average CO2 | | | 1,317 |
| Per Minute | | | |

Scenario 2: With Calcium Hydroxide Solution:

TABLE 33
Sealant with Calcium Hydroxide Solution

| | | | 1^{st} Reading | 2^{nd} Reading | Average |
|---|---|---|---|---|---|
| Parameter | Value | | (ppm) | (ppm) | (ppm) |
| Sealant | 20 | g | | | |
| Solution | 5 mL (5 g/L | | | | |
| | solution) | | | | |
| Area | 8 × 25 | cm | | | |

| | | | |
|---|---|---|---|
| Reading A | 738 | 721 | 730 |
| Reading B | 714 | 692 | 703 |
| Reading C | 688 | 668 | 678 |
| Average CO2 | | | 704 |
| Per Minute | | | |

Scenario 3: Calcium Hydroxide Powder and Water

TABLE 34
Sealant without Calcium Hydroxide

| | | | | |
|---|---|---|---|---|
| | | 1^{st} Reading | 2^{nd} Reading | Average |
| Parameter | Value | (ppm) | (ppm) | (ppm) |

| | | |
|---|---|---|
| Sealant | 20 | g |
| Powder | 5 | g |
| Water | 5 | mL |
| Area | 8 × 25 | cm |

| | | | |
|---|---|---|---|
| Reading A | 725 | 693 | 709 |
| Reading B | 650 | 585 | 618 |
| Reading C | 585 | 486 | 536 |
| Average CO2 | | | 621 |
| Per Minute | | | |

Accordingly, a crack filler employed with a solution of calcium hydroxide according to an embodiment of the invention reduced the carbon dioxide level for the area of 200 cm² from 1,317 ppm per minute to 704 ppm per minute. Replacing the solution with a powder and some water reduced the carbon dioxide level to 621 ppm per minute. Accordingly, a product according to an embodiment of the invention deployed in conjunction with a sealant, at the ratios outline or at ratios, enabled the capture of carbon dioxide

Table 35 outlines the results of extending the measurements to 24 hours after deploying the sealant by which time it had cured to a solid. The compositions, area, weights etc. being as given by Tables 32 to 34.

TABLE 35
Test Results after 1 Hour and 24 Hours

Carbon Dioxide Concentration

| | After 1 hr | After 24 hrs |
|---|---|---|
| Test | (Sealant Wet) | (Sealant Dry) |
| Without Calcium Hydroxide | 1,317 | 1,276 |
| With Calcium Hydroxide | 704 | 927 |
| Solution | | |
| With Calcium Hydroxide | 621 | 640 |
| Powder | | |

Table 36 below outlines experimental results for used car oil and roof sealants for carbon dioxide levels prior to the addition of a powder or solution according to embodiments of the invention. Table 37 presents the same materials but now combined with different powders according to embodiments of the invention. It is therefore evident that the embodiments of the invention are able to reduce carbon dioxide from a roof sealant product where the powder comprises calcium hydroxide, activated carbon powder or a combination of the two. Similarly, the used car oil can be processed to reduce carbon dioxide by using a powder according to an embodiment of the invention comprising calcium hydroxide to provide an alkaline solution in combination with the used car oil for a saponification reaction. The powder according to embodiments of the invention may or may not also contain activated carbon powder.

TABLE 36
Carbon Dioxide Measurements without
Embodiments of the Invention

| | | CO₂ after | CO₂ after | CO₂ after |
|---|---|---|---|---|
| | | 1 hour | 24 Hours | 9 days |
| Test | Test Components | (ppm) | (ppm) | (ppm) |
| 9 | Used car oil (5 mL) | 726 | 777 | — |
| 8 | Roof sealant (20 g) | 994 | 740 | 1381 |
| 2 | Used car oil (5 mL) + | 1,260 | 687 | 1,224 |
| | Roof sealant (20 g) | | | |

TABLE 37
Carbon Dioxide Measurements with Embodiments of the Invention

| | | CO₂ after | CO₂ after | CO₂ after |
|---|---|---|---|---|
| | | 1 hour | 24 Hours | 9 days |
| Test | Test Components | (ppm) | (ppm) | (ppm) |
| 1 | Used car oil (5 mL) + Calcium Hydroxide (3 g) | 659 | 793 | 630 |
| 10 | Roof sealant (20 g) + Calcium Hydroxide (3 g) | 569 | 571 | — |
| 3 | Roof sealant (20 g) + Active carbon powder (3 g) | 632 | 751 | 733 |
| 4 | Used car oil (5 mL) + Active carbon powder (3 g) | 683 | 595 | 668 |
| 5 | Used car oil (5 mL) + Roof sealant (20 g) + Active | 716 | 664 | 694 |
| | carbon powder (3 g) | | | |
| 6 | Used car oil (5 mL) + Roof sealant (20 g) + | 633 | 740 | 715 |
| | Calcium Hydroxide (3 g) | | | |
| 7 | Used car oil (5 mL) + Roof sealant (20 g) + | 724 | 657 | 700 |
| | Calcium Hydroxide (3 g) + Active carbon powder | | | |
| | (3 g) | | | |

Within the tests performed, calcium hydroxide powder and activated carbon powder were used as an additive for mixture with roof sealant and recycling of used car oil. From Table 36, it can be seen that:
Test 8 with roof sealant only shows a high pollution CO2 reading long-term;
Test 9 with used car oil only showed a lower pollution CO2 reading than roof sealant alone because used car oil contains carbon (carbonated oil), which works similarly as activated carbon, thereby reducing CO2 readings; and
Test 2 where used car oil was mixed with roof sealant, the CO2 levels appeared high short-term and long-term, after an hour and after 9 days, respectively.

However, when referring to Table 37 where all experiments included the step of adding additive powders (active carbon powder and calcium hydroxide), it is evident that these helped reduce carbon dioxide released by the mixture. In fact, all of the tests, test 1, 3, 4, 5, 6, 7, and 10 all produced significant reductions in CO2 levels over short-term and long-term timeframes.

Accordingly, it can be seen that in both instances the carbon dioxide levels remain lower compared to the sealant without a solution or powder according to an embodiment of the invention. It would be evident that the sealant may be employed with the powder/solution pre-mixed or applied after dispensing. Potentially, the solutions/powders according to embodiments of the invention may be employed with any material that is spread, sprayed, painted, dispensed containing petroleum products to reduce carbon dioxide emissions from the deployed material.

With respect to the saponification process, we see from Equations (14A) and (14B) that a soap can be formed from an oil or fat which is presented as tri-glycerin by way of an example.

Tri-Glycerin+Alkaline→Triple Alcohol+Soap (14A)

CH₂—O—CO—R CH₂—OH

CH₂—O—CO—R+3NaOH→CH₂—OH+3R—COONa (14B)

CH₂—O—CO—R CH₂—OH

Accordingly, if we exchange sodium hydroxide (NaOH) with calcium hydroxide (Ca(OH)2), we obtain Equations (15A) and (15B).

Tri-Glycerin+Alkaline→Triple Alcohol+Soap (15A)

CH₂—O—CO—R CH₂—OH

CH₂—O—CO—R+3Ca(OH)₂→CH₂—OH+3(R—COO)₂Ca (15B)

CH₂—O—CO—R CH₂—OH

In this manner, we can generate a saponification reaction with an oil, e.g. used car oil. Optionally, the calcium hydroxide may be sodium hydroxide (NaOH) or potassium hydroxide (KOH) within other embodiments of the invention or a combination of two or more of these hydroxides. The saponification generates a soap which is a salt of a fatty acid, which in turn is a carboxylic acid with carbon chains. The saponification reaction can remove at least 18 carbon molecules because a soap molecular formula is actually C₁₇H₃₅COO. Within the art, a “Saponification Number” is the number of milligrams of an alkaline, such as sodium hydroxide, potassium hydroxide or calcium hydroxide, required to saponify one gram of fat under the conditions specified. Accordingly, the inventors performing the tests titrated a calibrated amount of oil with a predetermined volume of alkaline solution according to the pH in the presence of an indicator solution for calibration. Accordingly, the inventors' experiments were performed using recycled car oil and roof sealant with calcium hydroxide and obtained good results.

### 9: Extending Lifetime of Cut Flowers

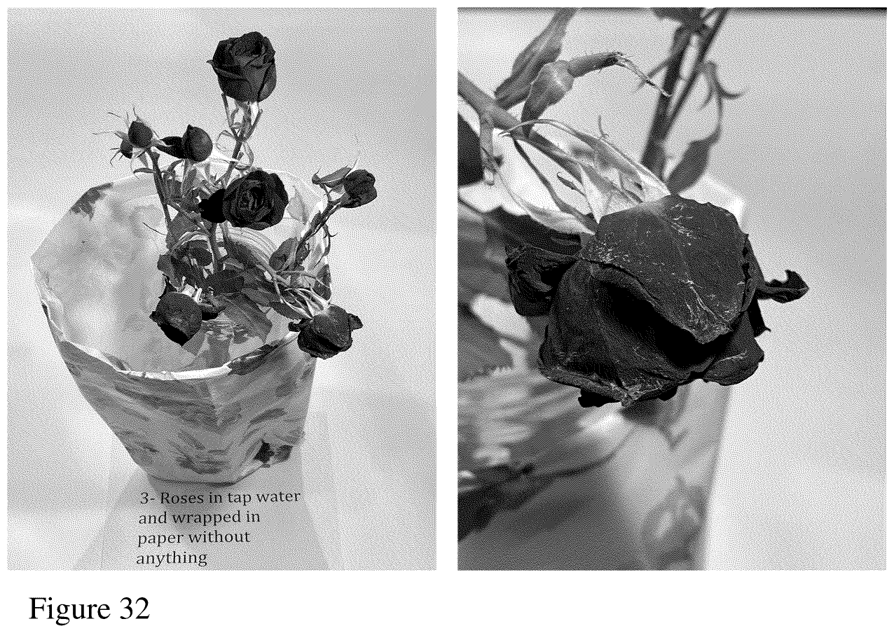

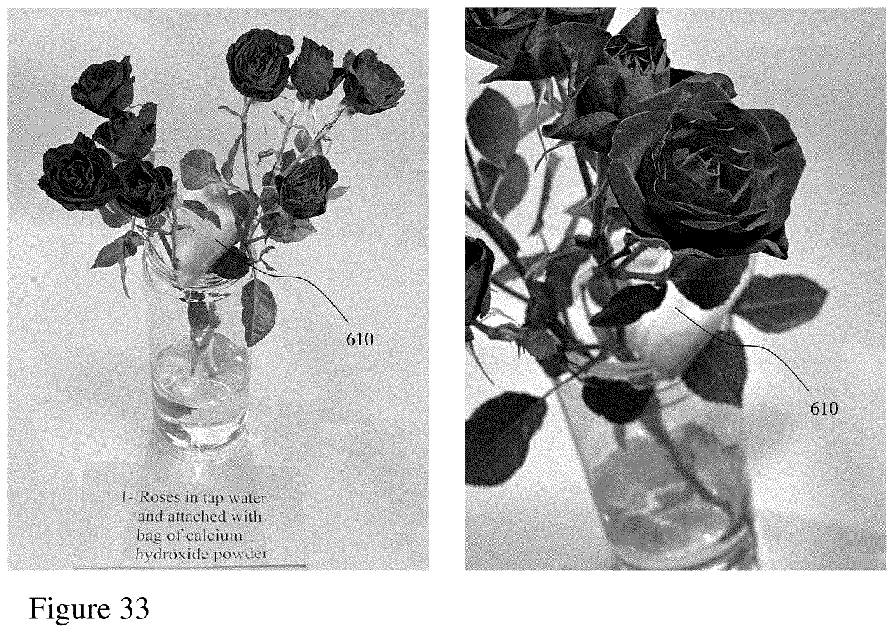

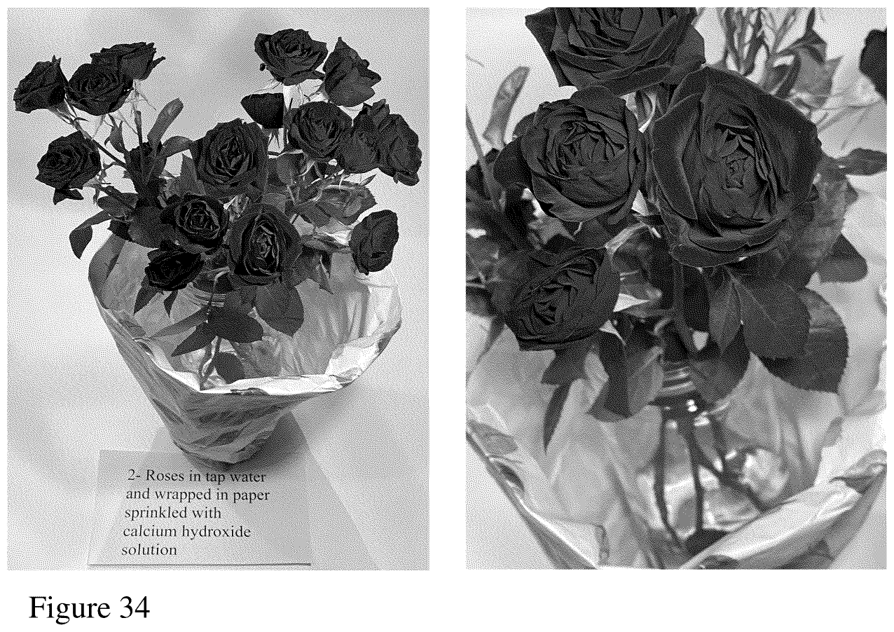

According to an embodiment of the invention, the inventors have established that a solution or powder may be employed to extend the lifetime and freshness of cut flowers. Experiments performed by the inventors have shown an increase in lifetime by the addition of calcium hydroxide to absorb carbon dioxide in the vicinity of the flowers. Referring to FIG. 32, there are depicted photographs of roses 1 week after purchase without any additional treatment showing that the roses are “spoiling” as the outer petals on each flower start decaying and some flowers droop. In contrast, in FIG. 33 there are depicted photographs of roses 1 week after purchase with a Product Package, Housing 610, according to an embodiment of the invention placed in the vicinity of the roses where the roses are clearly evident as being in good condition. Similarly, in FIG. 34 there are depicted photographs of roses 1 week after purchase where paper wrapping around the roses was soaked with a solution according to an embodiment of the invention.

Accordingly, a Product Package may be employed in conjunction with cut flowers during their storage and display by retailers/producers etc. as well as by consumers upon purchase of the cut flowers. In some embodiments of the invention, the cut flowers are sold with the Product Package attached to the cut flowers (for example by an elastic band, twist-tie, etc.) or attached to wrapping associated with the cut flowers wherein it can be removed and attached to a vase or other vessel within which the cut flowers are placed. Optionally, the Product Package may comprise an adhesive patch with removable cover allowing the Product Package to be attached to the vessel the cut flowers are placed in.

Within the scenario depicted in FIG. 34, the wrapping (e.g. plastic, paper, or plastic coated paper) employed to wrap the cut flowers for storage, display, transportation etc. may be sprayed with a solution according to an embodiment of the invention. Alternatively, a transparent or coloured plastic sheet may be sold separately or provided with cut flowers which is coated in the solution for use in conjunction with a vessel the cut flowers are placed within.

Within the foregoing disclosure of the exemplary embodiments of the present invention has been presented for purposes of illustration and description where exemplary compositions, mixtures and solutions have been presented together with exemplary ratios of components of said compositions, mixtures and solutions. However, it would be evident to one of skill in the art that other ratios of the constituents of said compositions, mixtures and solutions may be employed within embodiments of the invention without departing from the scope of the invention.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

1. The method according to claim 34, comprising:
executing the first process with respect to a quantity of produce; wherein
the first process delays at least one of a spoiling and a ripening of the produce.

2. The method according to claim 1, wherein
the produce comprises one of a fruit, a vegetable and an herb; and
the first process comprises applying at least one of a solution and a powder to the produce comprising calcium hydroxide;
the at least one of the solution and the powder absorbs at least one of carbon dioxide in the vicinity of the produce via a first chemical reaction and sulphur dioxide in the vicinity of the produce via a second chemical reaction.

3. The method according to claim 1, wherein
the produce comprises one of a fruit, a vegetable and an herb; and
the first process comprises applying at least one of a solution and a powder to the produce comprising calcium hydroxide and pectin extract;
the at least one of the solution and the powder absorbs at least one of carbon dioxide in the vicinity of the produce via a first chemical reaction and sulphur dioxide in the vicinity of the produce via a second chemical reaction;
when applied at least one of the solution and the powder forms a thin film over a portion of the produce; and
the pectin acts as a food preservative.

4. The method according to claim 1, wherein
the produce comprises one of a fruit, a vegetable and an herb; and
the first process comprises applying at least one of a solution and a powder to the produce comprising calcium hydroxide and a gelling agent;
the solution absorbs at least one of carbon dioxide in the vicinity of the produce via a first chemical reaction and sulphur dioxide in the vicinity of the produce via a second chemical reaction;
the solution once applied forms a thin film upon the produce; and
the thin film prevents at least one of a pollutant and a bacteria within the environment contacting the outer surface of the produce over that portion of the produce covered by the solution.

5. The method according to claim 1, wherein
the produce comprises one of a fruit, a vegetable and an herb; and
the first process comprises applying at least one of a solution and a powder to the produce comprising calcium hydroxide and a gelling agent;
the solution absorbs carbon dioxide in the vicinity of the produce via a chemical reaction;
the solution once applied forms a thin film upon the produce; and
the step of applying comprises at least one of spraying and dipping.

6. The method according to claim 1, wherein
the produce comprises one of a fruit, a vegetable and an herb;
the first process comprises storing the produce in association with a container ;
the container comprises:
a casing;
a permeable membrane forming a predetermined portion of the casing; and
calcium hydroxide powder disposed within the container; and
carbon dioxide released by the produce enters the container through the permeable membrane and is absorbed by the calcium hydroxide powder via a chemical reaction.

7. The method according to claim 6, wherein
the permeable membrane allows a further gas within the environment surrounding the produce to pass through from the environment into the casing; and
the further gas reacts with the calcium hydroxide powder within the container.

8. The method according to claim 6, wherein
storing the produce in association with the container comprises one of:
laying the produce upon the container such that the permeable membrane of the container is disposed towards the produce;
placing the produce within the container such that the permeable membrane of the container is disposed towards the produce; and
wrapping the produce within the container such that the permeable membrane of the container is disposed towards the produce.

9. The method according to claim 1, wherein
the produce comprises one of a fruit, a vegetable and an herb; and
the first process comprises storing the produce in association with a container comprising:
a casing;
a permeable membrane forming a predetermined portion of the casing;
calcium hydroxide powder disposed within the container; and
activated carbon disposed within the container; and
carbon dioxide released by the produce enters the container through the permeable membrane and is absorbed by the calcium hydroxide powder via a chemical reaction.

10. The method according to claim 9, wherein
storing the produce in association with the container comprises one of:
laying the produce upon the container such that the permeable membrane of the container is disposed towards the produce;
placing the produce within the container such that the permeable membrane of the container is disposed towards the produce; and
wrapping the produce within the container such that the permeable membrane of the container is disposed towards the produce.

11. The method according to claim 1, wherein
the produce comprises one of a fruit, a vegetable and an herb; and
the first process comprises storing the produce in association with a container comprising:
a casing;
a permeable membrane forming a predetermined portion of the casing; and
calcium hydroxide powder disposed within the container;
carbon dioxide released by the produce enters the container through the permeable membrane and is absorbed by the calcium hydroxide powder via a chemical reaction; and
the container forms a predetermined portion of a storage container within which the produce is disposed.

12. The method according to claim 11, wherein
the permeable membrane allows a further gas within the environment surrounding the produce to pass through from the environment into the casing; and
the further gas reacts with the carbon hydroxide powder within the container.

13. The method according to claim 11, wherein
storing the produce in association with the container comprises one of:
laying the produce upon the container such that the permeable membrane of the container is disposed towards the produce;
placing the produce within the container such that the permeable membrane of the container is disposed towards the produce; and
wrapping the produce within the container such that the permeable membrane of the container is disposed towards the produce.

14. The method according to claim 1, wherein
the produce comprises one of a fruit, a vegetable and an herb; and
the first process comprises storing the produce in association with a container comprising:
a casing;
a permeable membrane forming a predetermined portion of the casing;
calcium hydroxide powder disposed within the container; and
activated carbon disposed within the container;
carbon dioxide released by the produce enters the container through the permeable membrane and is absorbed by the calcium hydroxide powder via a chemical reaction; and
the container forms a predetermined portion of a storage container within which the produce is disposed.

15. The method according to claim 14, wherein
storing the produce in association with the container comprises one of:
laying the produce upon the container such that the permeable membrane of the container is disposed towards the produce;
placing the produce within the container such that the permeable membrane of the container is disposed towards the produce; and
wrapping the produce within the container such that the permeable membrane of the container is disposed towards the produce.

16. The method according to claim 1, wherein
the first process comprises at least one of applying a solution comprising calcium hydroxide to the produce or applying the solution to a sheet which it then wrapped around the produce; wherein
the solution is applied to the at least one of the produce and the sheet by at least one of dipping, spraying, misting, and coating.

17. The method according to claim 34, whereim
the first process relates to at least one of storing and shipping at least one of a plurality of seeds and a plurality of seedlings with container;
the container comprises:
a casing;
a permeable membrane forming a predetermined portion of the casing; and
calcium hydroxide powder disposed within the container; and
the calcium hydroxide absorbs at least one of moisture and carbon dioxide within the vicinity of the plurality of seeds and the plurality of seedlings.

18. The method according to claim 17 wherein
the reduction in at least one of moisture and carbon dioxide within the vicinity of the plurality of seeds and the plurality of seedlings reduces at least one of a rate of germination of the plurality of seeds and a rate of root spoilage of the plurality of seedling.

19. The method according to claim 34, whereiffi
the first process relates to an additive for an organic byproduct;
the additive comprises a calcium hydroxide solution and coffee grounds in a first predetermined ratio;
the additive is added to the organic byproduct at a second predetermined ratio; and
the organic byproduct with additive is intended for application to soil.

20. The method according to claim 19, wherein
the additive further comprises an acid for adjusting a pH of either the additive or the additive combined with a fluid.

21. The method according to claim 20, wherein
the fluid is water.

22. The method according to claim 19, wherein
the organic byproduct is one of dry manure, liquid manure and compost;
the first predetermined ratio is 1 kilogram of coffee grounds per 4 liters of calcium hydroxide solution;
the second predetermined ratio is 4 kilograms of organic byproduct to each kilogram of coffee grounds when the organic byproduct is dry manure or compost and 4 liters of organic byproduct to each kilogram of coffee grounds when the organic byproduct is liquid manure; and
the calcium hydroxide solution comprises 2 kilograms of calcium hydroxide per 1,000 liters of water.

23-29. (canceled)

30. The method according to claim 34, wherein
the first process relates to reducing emissions from a diesel engine comprising:
injecting a solution at specific flow rate into an exhaust of the diesel engine when the diesel engine is running; wherein
the solution comprises urea, water and calcium hydroxide.

31. The method according to claim 30, wherein
the solution reduces emissions of nitrogen oxides (NOx) from the exhaust of the diesel engine via a reaction with the NOx; and
the calcium hydroxide reduces emissions of at least one of:
carbon dioxide (CO2) via a first reaction with the CO2 from the exhaust of the diesel engine;
and sulphur dioxide (SO2) via second reaction from the exhaust of the diesel engine

32. The method according to claim 34, whereim
the first process relates to at least one of extending freshness and delaying ripening of produce with calcium hydroxide; and
the calcium hydroxide is applied by at least one of spraying a calcium hydroxide solution onto the produce; misting the calcium hydroxide solution onto the produce, dipping the produce into the calcium hydroxide solution; storing the produce with calcium hydroxide powder, and coating the produce with calcium hydroxide powder.

33. The method according to claim 32, wherein
the calcium hydroxide is applied in conjunction with at least one activated carbon and pectin.

34. A method comprising:
employing calcium hydroxide within a first process to effect a change within an environment.

35. The method according to claim 34, wherein
the first process relates to reduction of at least one of coronavirus cells, a bacterial infection and a fungal infection upon a surface of the environment;
the calcium hydroxide is applied as part of a powder or a solution.

36. The method according to claim 34, wherein
the first process relates to reduction of at least one of carbon dioxide and sulfur dioxide;
the calcium hydroxide is added as either a solution or a powder to a petroleum product; and
the petroleum product is one of a sealant and a caulking agent.

37. The method according to claim 34, wherein
the first process relates to the manufacture of an animal feed where the calcium hydroxide is an additive added to one or more animal feed components in either a first configuration or a second configuration;
in the first configuration the calcium hydroxide is added to one or more animal feed components discretely to react with carbon dioxide generated by an animal digesting the animal feed such that the carbon dioxide reacts with the calcium hydroxide and expelled as a precipitate within excrement produced by the animal such that the change in the environment is a reduction in carbon dioxide released into atmosphere; and
in the second configuration
the calcium hydroxide is added to one or more animal feed components with an oxidizing agent such that:
the calcium hydroxide reacts with carbon dioxide generated by an animal digesting the animal feed to generate a precipitate which is expelled within the excrement produced by the animal; and
the oxidizing agent reacts with methane generated by the animal digesting the animal feed and the carbon dioxide generated as a product of oxidising the methane reacts with the calcium hydroxide to generate a precipitate which is expelled within the excrement produced by the animal; and
the change in the environment is a reduction in both carbon dioxide and methane released into atmosphere.

38. A method comprising:
reacting carbon dioxide and hydrogen to generate formic acid (CH₂O₂);
reacting the formic acid with methane and water within a defined temperature range in the presence of a bacteria to generate acetic acid (C₂H₄O₂) and methane.
